(19) United States
(12) Patent Application Publication
(10) Pub. No.: US 2018/0338945 A1
(43) Pub. Date: Nov. 29, 2018

(54) SKIN ADHESIVES, ANTIMICROBIAL COMPOSITIONS, ARTICLES, AND METHODS FOR THE USE THEREOF

(71) Applicant: DERMALINK TECHNOLOGIES, INC., Pennington, NJ (US)

(72) Inventor: Mahesh Sambasivam, Pennington, NJ (US)

(21) Appl. No.: 15/963,356

(22) Filed: Apr. 26, 2018

Related U.S. Application Data
(63) Continuation of application No. PCT/US2016/059273, filed on Oct. 28, 2016.
(60) Provisional application No. 62/249,222, filed on Oct. 31, 2015.
(60) Provisional application No. 62/304,786, filed on Mar. 7, 2016.
(60) Provisional application No. 62/328,678, filed on Apr. 28, 2016.

(51) Int. Cl. A61K 31/215; A61K 47/38; A61K 47/10; A61K 9/06; A61K 9/00; A61P 17/02; A61P 31/04; A61P 31/12

(52) U.S. Cl. CPC A61K 31/215; A61K 47/38; A61K 47/10; A61P 31/12; A61K 9/0014; A61P 17/02; A61P 31/04; A61K 9/06

(57) ABSTRACT

The present disclosure relates to skin adhesives, antimicrobial compositions, and articles thereof, including methods and processes of forming such antimicrobial compositions, medical devices, and articles thereof. The antimicrobial compositions include, for example, adhesive compositions, gels, cleansers, wound dressings and foams.

### RELATED APPLICATIONS

This application is a continuation of International Application No. PCT/US2016/59273, which designated the United States and was filed on Oct. 28, 2016, published in English, which claims the benefit of U.S. Provisional Application No. 62/249,222, filed on Oct. 31, 2015, U.S. Provisional Application No. 62/304,786, filed on Mar. 7, 2016, and U.S. Provisional Application No. 62/328,678 filed on Apr. 28, 2016. The entire teachings of the above applications are incorporated herein by reference.

### FIELD OF INVENTION

The present disclosure relates to adhesives, antimicrobial compositions including, for example, antimicrobial adhesives, antimicrobial films, and antimicrobial foams, and dressings. The disclosure also relates to medical devices, articles, methods and processes for the use of any of thereof.

### BACKGROUND OF THE INVENTION

In healthcare and in wound care, it is important to keep patients and caregivers free of infection. In patients with wounds, it is also important to facilitate the wound healing process. Wound dressings are widely used to protect wounds from external factors and to maintain a moist environment which is required for the healing process by managing the wound exudate. In addition, these dressings may contain active agents such as antimicrobial agents, wound healing agents, growth factors, etc., to reduce the bio-burden in the wound bed, and also to speed up the healing process. Wound dressings are coated, laminated or impregnated with the active agents to promote healing or reduce infection in a wound bed.

U.S. Patent Application Publication No. 2013/0101633A1 discloses an antimicrobial silicone gel adhesive composition comprising silver and its salts, and hydrophilic additive that swells the adhesive. There are also commercial products in the market with antimicrobial agents in foam pads or gels, for example, BIOPATCH® protective disk (from Ethicon), TEGADERM™ CHG Dressings (3M), wherein a gel or foam pad with the antimicrobial agent chlorhexidine gluconate (CHG), is provided with a window dressing. The antimicrobial is limited to the pad or foam area but may also be incorporated in the window dressing. In addition, SURGICLEAR™ Antimicrobial Clear Silicone Surgical Dressing from Covalon Technologies Ltd., (SurgiClear is a Trademark of Covalon Technologies Ltd.) is a clear antimicrobial silicone surgical dressing with both silver and chlorhexidine as dual antimicrobial agents.

Commercially available scaffolds such as collagen dressings, for example, PURAPLY™ Antimicrobial from Organogenesis Inc., and Puracol Plus AG+ from Medline Industries, Inc. are also available and use polyhexamethylene biguanide and silver, respectively as the antimicrobial agents. These dressings are used as a scaffold for ulcers, slow to heal wounds, partial to full thickness wounds, and other wounds as indicated.

There remains a need in the art for additional adhesives, antimicrobial compositions, cleansers, gels, foam compositions, scaffolds, and methods for the use thereof.

### SUMMARY OF THE INVENTION

One objective of the invention is to provide antimicrobial compositions for medical applications utilizing antimicrobial agents that are not toxic or hazardous to mammalian tissue and/or skin. Such antimicrobial compositions include, for example, adhesive compositions, gels (such as wound gels) and cleansers. Another objective is to provide adhesive, film, gel, cleanser, and foam compositions that have antimicrobial properties including such agents. Yet another objective relates to methods and processes of preparing antimicrobial adhesives, films, layers on surfaces, articles, including medical devices. Yet additional objectives are directed to method of treating a wound comprising administering an antimicrobial agent and/or antimicrobial composition described herein. A further objective is to provide improved adhesive formulations, wound dressings including said agents and methods for the use thereof. The above objectives are met by compositions, adhesives, films, medical devices, and methods described herein.

In aspects, the compositions of the present disclosure may be used against one or more infection-associated bacteria, fungi, or yeasts present in a wound environment, hospitals, medical devices, surgical sites, biofilms, and the like. The compositions may be effective against microbes including, but not limited, gram-positive, gram-negative, yeast, mold, spores, antibiotic-resistant strains, and the like.

In an aspect of the present disclosure, the invention encompasses an antimicrobial adhesive composition, including at least one antimicrobial agent and at least one adhesive. The adhesive may be a pressure sensitive adhesive and/or gel adhesive, and may be suitable to secure medical devices to mammalian body, skin, tissue, mucosal tissue, and the like. In certain embodiments, the adhesive is a gel adhesive. In an additional aspect, the antimicrobial adhesive composition includes at least two antimicrobial agents and at least one adhesive.

The antimicrobial agents of the present disclosure may be selected from the group consisting of natural polypeptides, N-acylamino acid esters and/or their salts, esters of glycerol and saturated and/or unsaturated fatty acids (C₆-C₂₀), saturated and/or unsaturated alcohols with C₆-C₂₀ carbon atoms, saturated and/or unsaturated long chain alcohols (C₆-C₂₀), and combinations thereof. In certain embodiments, the antimicrobial agent is a N^{α-}lauroyl arginine ester or a salt thereof, including, for example, N^{α}-lauroyl-arginine ethyl ester or a salt thereof.

The adhesives of the present disclosure may be selected from: silicones and/or their copolymers, polyvinylmethyl ether and/or its copolymers, polyacrylates and/or their copolymers, polymethacrylates and/or their copolymers, polyacrylic acid and/or its copolymers, styrenic rubbers, polyvinylpyrrolidone and/or its copolymers, polyvinyl alcohol and/or its copolymers, polyurethanes, polyolefins, and combinations thereof.

In certain aspects, the antimicrobial adhesive composition comprises a silicone gel adhesive, a N^{α}-lauroyl-arginine ester or a salt thereof (preferably, a N^{α}-lauroyl-arginine ethyl ester or a salt thereof), and a non-ionic additive. In additional aspects, antimicrobial adhesive composition comprises:
a. a silicone gel adhesive in an amount of about 75 to about 95% by weight, wherein the silicone gel adhesive is prepared via hydrosilylation in the presence of a platinum catalyst;
b. a N^{α}-lauroyl-arginine ester or a salt thereof in an amount of about 0.5 to about 10% by weight; and
c. a non-ionic additive, wherein the non-ionic additive is present in an amount of about 0.5 to about 10% by weight.

The non-ionic additive can, for example, be a non-ionic hydrocolloid. In yet further aspects, the non-ionic additive is a cellulose. In yet additional aspects, the non-ionic additive is selected from the group consisting of hydroxyethyl cellulose, hydroxypropyl cellulose, methyl cellulose, carboxymethylcellulose, maltodextrin, dextran, xanthan gum, guar gum, pectin, beta-glucans, rice protein, oat protein, potato protein, and polylysine. The N^{α}-lauroyl-arginine ester or a salt thereof can be N^{α}-lauroyl-arginine ethyl ester or a salt thereof, for example, the hydrochloride salt of N^{α}-lauroyl-arginine ethyl ester.

In additional aspects, the invention encompasses a method of preparing an antimicrobial adhesive composition comprising a silicone gel adhesive and a N^{α}-lauroyl-arginine ester or a salt thereof, the method comprising:
a. preparing a mixture comprising an alkenyl and/or alkynyl-substituted polydiorganosiloxane, a polydiorganosiloxane comprising silicon-bonded hydrogen atoms, a platinum catalyst, N^{α}-lauroyl-arginine ester or a salt thereof, and a non-ionic additive; and
b. curing the above mixture from (a) on a carrier.

In certain aspects, the non-ionic additive is selected from the group consisting of hydroxyethyl cellulose, hydroxypropyl cellulose, methyl cellulose, carboxymethylcellulose, maltodextrin, dextran, xanthan gum, guar gum, pectin, beta-glucans, rice protein, oat protein, potato protein, and polylysine. Examples of carriers include, but are not limited to, a polymer film, non-woven, woven fabric, mesh, foam, gel, and a combination thereof.

The compositions described herein may further include one or more components selected from the group consisting of: solvent, pH-buffering agents, stabilizing agents, surfactants, antibiotics, wound healing agents, hormones, growth factors, and combinations thereof.

The antimicrobial adhesive compositions described herein can provide the benefit of securing medical devices to the human body or the skin, and maintaining effective antimicrobial activity. Skin adhesives are widely used in wound dressings, fixation tapes, burn management, vacuum therapy, ostomy appliances, and the like. Use of the antimicrobial adhesive compositions of the present disclosure provides the dual effect of adhesive property along with antimicrobial activity. Since the compositions do not include cytotoxic compounds, they are safe for use on mammalian body, internal and external wounds, medical devices, surgical and hospital environment.

In certain additional aspects, the antimicrobial adhesive composition may include a combination of ε-polylysine, N^{α}-lauroyl-arginine ethyl ester hydrochloride, and an adhesive, wherein the adhesive may be a silicone adhesive. For example, the composition can comprise: antimicrobial adhesive composition comprises:
a. a silicone gel adhesive in an amount of about 75 to about 95% by weight, wherein the silicone gel adhesive is prepared via hydrosilylation in the presence of a platinum catalyst;
b. a N^{α}-lauroyl-arginine ester or a salt thereof in an amount of about 0.5 to about 10% by weight; and
c. polylysine (for example, ε-polylysine) in an amount of about 0.5 to about 10% by weight.

In further aspects, the antimicrobial adhesive composition can include a combination of an ester of glycerol and lauric acid, and an adhesive, wherein the adhesive is a silicone adhesive. In further aspects, the antimicrobial adhesive composition can include a combination of ester of glycerol and lauric acid, ε-polylysine, and an adhesive, wherein the adhesive can be a silicone adhesive. In further aspects, the antimicrobial adhesive composition can include a combination of ester of glycerol and lauric acid, N^{α}-lauroyl-arginine ethyl ester hydrochloride, and an adhesive, wherein the adhesive is a silicone adhesive.

In aspects, in the antimicrobial adhesive composition, the adhesive can be present at 10.0-90.0 wt %, or 20.0-80.0 wt %, or 40.0-70.0 wt %, or the like of the weight of the composition. The amount of adhesive in the composition according to the present disclosure may be determined by the amount of adhesiveness and/or tackiness may be required for the application.

In additional aspects, the antimicrobial agent is present in sufficient amount to be effective as an antimicrobial composition in wounds, medical devices, surfaces, components, skin, and the like. The antimicrobial agent may be present in the range of 0.5-90.0 wt %, 5.0-80.0 wt %, or 20.0-70.0 wt %, or the like of the weight of the composition.

In aspects, the antimicrobial adhesive composition can be delivered as a solution, a paste, a gel, a tape, a film, an adhesive, a layer, a non-perforated sheet, a perforated sheet, a foam, a woven material, a non-woven material, a fiber, a porous membrane, a non-porous membrane, and combinations thereof.

In aspects, the antimicrobial adhesive composition comprises a silicone adhesive wherein the silicone adhesive comprises at least one alkenyl- and/or alkynyl-substituted polysiloxane, at least one polysiloxane comprising silicon-bonded hydrogen atoms, and at least one hydrosilylation catalyst and/or a peroxide catalyst. In aspects, the silicone adhesive comprises at least one alkenyl- and/or alkynyl-substituted polysiloxane covalently crosslinked to the at least one polysiloxane comprising silicon-bonded hydrogen atoms, thereby forming an adhesive.

In further aspects, the antimicrobial adhesive composition comprises at least one polyorganosiloxane, and at least one silicate resin.

In certain additional aspects, the antimicrobial adhesive composition described herein does not include a silicate resin.

In further aspects, the antimicrobial adhesive composition comprises a silicone adhesive, wherein the silicone adhesive comprises at least one hydroxyl-terminated polyorganosiloxane, at least one silane, and at least one condensation cure catalyst.

In yet further aspects, the antimicrobial adhesive composition comprises a silicone adhesive, wherein the silicone adhesive comprises at least one copolymer of 3-[tris(trimethylsilyloxy)silyl]propyl methacrylate (TRIS) and at least one acrylate and/or methacrylate. The acrylate is selected from n-butyl acrylate, t-butyl acrylate, octyl and/or iso-octyl acrylate, and/or ethylhexyl acrylate. The ratio of TRIS to acrylate or methacrylate may be modified to provide a copolymer with glass transition temperature below 25° C.

In further aspects, the antimicrobial adhesive composition may further include at least one additional antimicrobial agent with synergistic and/or enhanced antimicrobial activity. The presence of at least one additional antimicrobial agent improves the spectrum of activity against various microbes and/or enhances the activity of the composition. The additional antimicrobial agent may be selected from curcumin, 2-phenoxyethanol, tea tree oil (Melaleuca oil), natural oils, xylitol and its esters, lactoferrin, chlorhexidine salts, polymeric biguanides, non-polymeric biguanidines, hexetidine salts, quaternary ammonium compounds, cetylpyridinium salts, chloramine T, and metals including their oxides and salts, wherein the metal is selected from copper, zinc, and/or silver, and combinations thereof. The amount of the additional antimicrobial agent may be in the range of trace to 40.0 wt %, or trace to 30.0 wt %, or trace to 10.0 wt % of the total composition. In further aspects, the adhesive of the present disclosure may include a blend or mixture of the adhesives of the same chemistry or different chemistries as disclosed in the present disclosure. In further aspects, the antimicrobial adhesive composition according to the present disclosure may further include at least one additional antimicrobial agent with synergistic and/or enhanced antimicrobial activity.

In yet further aspects, the antimicrobial adhesive composition does not comprise an additional antimicrobial agent.

In further aspects, the antimicrobial adhesive composition according to the present disclosure can include one or more surfactants. The surfactants may facilitate the availability of the antimicrobial agent(s) to the site where the activity may be required such as wound surface. The surfactants according to the present disclosure may include cationic, anionic, nonionic, and/or amphoteric surfactants. The surfactants can, for example, include glycerols, silicone glycerol, silicone-polyether copolymers, polyalkylene oxides, quaternary ammonium salts, polysorbate, fatty acid esters of glycerol and other alcohols, sugar esters, alkyl sulfates, sulfosuccinates, and combinations thereof.

In aspects, the antimicrobial adhesive composition can include a hydrophilic additive. The hydrophilic additives may allow the composition to swell, dissolve, disperse, and/or gel in aqueous medium and/or physiological fluid. The hydrophilic additives can include citric acid and its salts, glycerols, glycerol esters, monosaccharides, disaccharides, oligosaccharides, polysaccharides, cellulose and its derivatives, hydrocolloids, polyalkylene oxides and their copolymers, polyvinyl alcohol and its copolymers, poly(vinyl pyrrolidone) and is copolymers, poly(vinylmethyl ether) and its copolymers, polymaleic anhydride copolymers, sulfonated polystyrene and its salts and/or copolymers, polyacrylamide and its copolymers, polyN-alkylacrylamide and its copolymers, sulfonated polyesters, polyacrylic acid and its copolymers, poly(N-isopropyl acrylamide) and its copolymers, polydimethlyamino methacrylate and its copolymers, gelatin, chitosan, hyaluronic acid, polyamides, polypeptides, polyvinyl amine, polyoxazoline and its copolymers, polyphosphazene and its copolymers, and combinations thereof.

In certain aspects, the antimicrobial composition is a cleanser, wherein the cleanser is an aqueous antimicrobial composition comprising:
a. N^{α}-lauroyl-arginine ester or a salt thereof (for example, N^{α}-lauroyl-arginine ethyl ester or a salt thereof) in an amount between about 0.01 to about 1% by weight of the composition; and
b. glycerol in an amount between about 0.1 and about 10% by weight of the composition.

In yet additional aspects, the antimicrobial composition is an antimicrobial wound gel comprising:
a. N^{α}-lauroyl-arginine ester or a salt thereof (for example, N^{α}-lauroyl-arginine ethyl ester or a salt thereof) in an amount between about 0.01 to about 3% by weight of the composition; and
b. a non-ionic thickener selected from the group consisting of hydroxyethylcellulose, hydroxypropyl cellulose, methyl cellulose, and polyethylene oxide in an amount between about 0.5 to about 5% by weight of the composition;

wherein the wound gel is an aqueous gel with a viscosity greater than 1,000 centipoise.

The invention also encompasses hydrophilic silicone gel adhesive compositions that can optionally further contain an antimicrobial agent. In some embodiments, the invention is directed to a hydrophilic silicone gel adhesive comprising:
a. polydimethylsiloxane in an amount of about 75 to about 95% by weight, wherein the polydimethylsiloxane is crosslinked by hydrosilylation in the presence of a hydrosilylation catalyst;
b. a non-ionic cellulose in an amount of about 1 to about 10% by weight; and
c. a plasticizing agent for the non-ionic cellulose in an amount of about 0.5 to about 20% by weight, wherein the plasticizing agent is selected from the group consisting of glycerol, glyceryl alkyl ether and glyceryl alkyl ester.

In an additional aspect, a method of preparing an adhesive or antimicrobial adhesive layer on a surface may include the steps of: i. preparing a mixture of the adhesive composition in accordance with the present disclosure; ii. optionally, adding at least one solvent and/or fluid to the mixture to form an intermediate mixture; iii. applying the mixture and/or the intermediate mixture to the surface to form a layer and; iv. curing, gelling, cooling, heating, radiating and/or drying the layer, thereby obtaining an antimicrobial adhesive layer on the surface. In aspects, the surface may be biological tissue, skin, film, foam, non-woven material, woven material, fabric, sheet, rubber, fibers, mesh, plastic, and combinations thereof.

In further aspects, the invention includes a method of delivering the adhesive or antimicrobial adhesive composition can include the steps of: preparing the composition in accordance with the present disclosure, and applying the preparation to the wound.

In another aspect, a method of delivering the adhesive or antimicrobial composition to a biofilm can include the steps of: preparing the antimicrobial composition in accordance with the present disclosure, and applying the preparation to the biofilm. In aspects, the biofilm may be present on a wound bed, tissue, and the like.

In aspects, the adhesive or antimicrobial composition, including, for example, the antimicrobial adhesive, the cleanser, the gel and the foam according to the present disclosure, can reduce the number of colony forming units (CFUs) of a microbe by at least one order of magnitude in 24 hours of exposure. In yet further aspects, the adhesive or antimicrobial composition, including, for example, the antimicrobial adhesive, the cleanser, the gel and the foam according to the present disclosure, can reduce the number of colony forming units (CFUs) of Staphylococcus aureus and Pseudomonas aeruginosa by at least one order of magnitude in 24 hours of exposure.

In further aspects, the invention encompasses a medical device including an antimicrobial layer, wherein the antimicrobial layer includes an antimicrobial adhesive composition in accordance with the present disclosure. In aspects, the medical device can, for example, be a catheter, a fixation tape, a cover dressing, an absorbent dressing, a needle, a tube, a surgical instrument, a tape, an implant, a mask, a scaffold, an ostomy appliance, a collection bag, and combinations thereof.

In yet further aspects, the invention includes a medical device including an adhesive layer, wherein the adhesive layer includes the hydrophilic silicone gel adhesive in accordance with the present disclosure. In aspects, the medical device can be a catheter, a fixation tape, a cover dressing, an absorbent dressing, a needle, a tube, a surgical instrument, a tape, an implant, a mask, a scaffold, an ostomy appliance, a collection bag, and combinations thereof.

In another aspect, the invention is directed to a wound dressing including a skin adhering region, wherein the skin adhering region includes an adhesive or antimicrobial adhesive composition in accordance with the present disclosure. The wound dressing may be a film dressing, a foam dressing, a hydrogel dressing, a hydrocolloid dressing, and the like. The skin adhering region may include the wound and/or tissue.

In yet an additional aspect, the invention includes a wound dressing comprising an absorbent region and a skin adhering region, wherein the absorbent region and/or the skin adhering region includes an antimicrobial adhesive composition in accordance with the present disclosure. The absorbent region may further include foams, fibers, nonwoven, hydrogel, and the like.

In an additional aspect, a wound dressing includes an antimicrobial adhesive composition, wherein the antimicrobial adhesive composition includes at least one antimicrobial agent, at least one adhesive, and at least one delivery agent, further the composition includes two phases including a continuous phase and a discontinuous phase, wherein the continuous phase may be an adhesive, and the discontinuous phase includes the antimicrobial agent(s) and the delivery agent, wherein the delivery agent breaks down in the wound or physiological environment to release the antimicrobial agent(s).

In aspects, the wound dressing according to the present disclosure comprises an antimicrobial agent, wherein the antimicrobial agent(s) may be selected from the group consisting of natural polypeptides, N-acylamino acid esters and/or their salts, esters of glycerol and saturated and/or unsaturated fatty acids (C₆-C₂₀), saturated and/or unsaturated alcohols with C₆-C₂₀ carbon atoms, and combinations thereof.

In yet additional aspects, the wound dressing comprises an antimicrobial agent wherein the antimicrobial agent is present in the range of 0.5-90.0 wt %, 5.0-80.0 wt %, or 10.0-70.0 wt %, or the like of the weight of the composition.

In aspects, the wound dressing comprises an adhesive, wherein the adhesive is selected from the group consisting of silicones and/or their copolymers, polyvinylmethyl ether and/or its copolymers, polyacrylates and/or their copolymers, polymethacrylates and/or their copolymers, polyacrylic acid and/or its copolymers, styrenic rubbers, polyvinylpyrrolidone and/or its copolymers, polyvinyl alcohol and/or its copolymers, polyurethanes, polyolefins, and combinations thereof.

In yet additional aspects the wound dressing comprises a hydrophilic silicone gel adhesive comprising:
a. polydimethylsiloxane in an amount of about 75 to about 95% by weight, wherein the polydimethylsiloxane is crosslinked by hydrosilylation in the presence of a hydrosilylation catalyst;
b. a non-ionic cellulose in an amount of about 1 to about 10% by weight; and
c. a plasticizing agent for the non-ionic cellulose in an amount of about 0.5 to about 20% by weight, wherein the plasticizing agent is selected from the group consisting of glycerol, glyceryl alkyl ether and glyceryl alkyl ester.

In some aspects, the wound dressing comprises an adhesive which is present in the range of 10.0-90.0 wt %, 20.0-70.0 wt %, or 40.0-60.0 wt % of the weight of the composition.

In aspects, the wound dressing comprises a delivery agent, wherein the delivery agent is hydrophilic, hydrophobic, amphiphilic, ionic, nonionic, amphoteric, and combinations thereof. In aspects, the delivery agent can include citric acid and/or its salts, glycerols, glycerol esters, polyalkylene oxides and their copolymers, monosaccharides, oligosaccharides, polysaccharides, polyvinyl alcohol and its copolymers, poly(vinyl pyrrolidone) and is copolymers, poly(vinylmethyl ether) and its copolymers, polymaleic anhydride copolymers, sulfonated polystyrene and its salts and/or copolymers, polyacrylamide and its copolymers, sulfonated polyesters, polyacrylic acid and its copolymers, poly(N-isopropyl acrylamide) and its copolymers, polydimethlyamino methacrylate and its copolymers, gelatin, chitosan, hyaluronic acid, polyamides, polypeptides, polyvinyl amine, polyoxazoline and its copolymers, polyphosphazene and its copolymers, hydrocolloids, surfactants, and combinations thereof.

In aspects, the wound dressing comprises a delivery agent wherein the delivery agent may be present in the range of 0.5-80.0 wt %, 1.0-60.0 wt %, or 10.0-50.0 wt %, of the weight of the composition.

In yet additional aspects, the wound dressing described can further include pH-buffering agent(s).

In certain aspects, an antimicrobial composition described herein can be used to treat an infection, a wound, and/or a biofilm. In aspects, the use of an antimicrobial adhesive composition described herein can be used to treat an infection, a wound, and/or a biofilm. In yet additional aspects, the antimicrobial composition described herein can be used to prevent an infection, a wound, and/or a biofilm.

In an additional aspect, the invention includes an antimicrobial film, non-woven, woven, gel, paste, or mesh including an antimicrobial composition, wherein the composition may include at least one antimicrobial agent and at least one oligomer and/or polymer, wherein the film, non-woven, woven, gel, paste or mesh may be impregnated, coated, blended, or treated with the antimicrobial composition. In other aspects, the monomer, oligomer, and/or polymer may also be capable of forming the film, non-woven, woven, gel, paste, or mesh. In aspects, the antimicrobial agent may be selected from: natural polypeptides, N-acylamino acid esters and/or their salts, esters of glycerol and saturated and/or unsaturated long chain acids (C6-C20), saturated and/or unsaturated long chain alcohols (C6-C20), and combinations thereof; wherein the oligomer and/or polymer may be selected from: silicones and/or their copolymers, polyvinylmethyl ether and/or its copolymers, polyacrylates and/or their copolymers, polymethacrylates and/or their copolymers, polyacrylic acid and/or its copolymers, and/or its salts, styrenic rubbers, polyvinylpyrrolidone and/or its copolymers, polyvinyl alcohol and/or its copolymers, polyurethanes, polycarbonates, polyamides and/or their copolymers, polyesters and/or their copolymers, polyolefins, polyvinyl chloride, polyethersulfone, polyether ether ketone (PEEK), and combinations thereof.

In aspects, the antimicrobial film, non-woven, woven, gel, paste, or mesh according to the present disclosure may inhibit the growth of Staphylococcus aureus and Pseudomonas aeruginosa by at least one order of magnitude in 24 hours according to the test disclosed in the present disclosure.

In aspects, the antimicrobial film, non-woven, woven, gel, paste, or mesh according to the present disclosure may inhibit the growth of Staphylococcus aureus and Pseudomonas aeruginosa in a zone of inhibition (ZOI) test, wherein the ZOI is at least equal to the size of the exposed film when tested according to the test disclosed in the present disclosure.

In aspects, the antimicrobial film, non-woven, woven, gel, paste, or mesh comprises a polymer and/or oligomer, wherein the polymer and/or oligomer may be present at 10.0-90.0 wt %, or 20.0-70.0 wt %, or 40.0-60.0 wt % of the weight of the composition.

The antimicrobial film, non-woven, woven, gel, paste, or mesh according to the present disclosure can comprise polymer and/or oligomer that is a silicone, wherein the silicone includes at least one alkenyl- and/or alkynyl-substituted polysiloxane, at least one polysiloxane comprising silicon-bonded hydrogen atoms, and at least one hydrosilylation catalyst and/or a peroxide catalyst.

The antimicrobial film, non-woven, woven, gel, paste, or mesh according to the present disclosure can comprise a polymer and/or oligomer that is a silicone, wherein the silicone includes at least one alkenyl- and/or alkynyl-substituted polysiloxane covalently crosslinked to the at least one polysiloxane comprising silicon-bonded hydrogen atoms.

In aspects, the antimicrobial film, non-woven, woven, gel, paste, or mesh according to the present disclosure, wherein the polymer and/or oligomer may be a silicone, wherein the silicone includes at least one polyorganosiloxane, and at least one silicate resin.

In aspects, the antimicrobial film, non-woven, woven, gel, paste, or mesh according to the present disclosure, wherein the polymer and/or oligomer may be a silicone, wherein the silicone includes at least one hydroxyl-terminated polyorganosiloxane, at least one silane, and at least one condensation cure catalyst.

In aspects, the antimicrobial film, non-woven, woven, or mesh according to the present disclosure, wherein the polymer and/or oligomer may be a silicone, wherein the silicone includes at least one copolymer of trimethylsiloxysilylpropyl acrylate and at least one acrylate, wherein the acrylate is selected from butyl acrylate, octyl and/or iso-octyl acrylate, and/or ethylhexyl acrylate.

In aspects, the antimicrobial film, non-woven, woven, gel, paste, or mesh according to the present disclosure, wherein the antimicrobial agent may be present in the range of 0.5-90.0 wt %, 5.0-80.0 wt %, or 10.0-70.0 wt %, of the weight of the composition.

In aspects, the invention includes the antimicrobial film, non-woven, woven, gel, paste, or mesh described herein wherein the composition may further include at least one additional antimicrobial agent with synergistic and/or enhanced antimicrobial activity. In aspects, the additional antimicrobial agent can be selected from curcumin, 2-phenoxyethanol, tea tree oil (Melaleuca oil), natural oils, xylitol and its esters, lactoferrin, chlorhexidine salts, polymeric biguanides, non-polymeric biguanidines, hexetidine salts, quaternary ammonium compounds, cetylpyridinium salts, chloramine T, and metals including their oxides and salts, wherein the metal may be selected from copper, zinc, and/or silver, and combinations thereof.

In aspects, the antimicrobial film, non-woven, woven, gel, paste, or mesh according to the present disclosure, wherein the composition may further include hydrophilic additives, surfactants, pH-buffering agents, and other pharmaceutically acceptable additives.

In another aspect, the invention includes a method of forming an antimicrobial film, non-woven, woven, or mesh according to the present disclosure, wherein the said method includes treating said film, non-woven, woven, gel, paste, or mesh with a powder, solution, dispersion, emulsion, and/or suspension of said antimicrobial composition of the present disclosure.

In aspects, the invention is a method of forming an antimicrobial film, non-woven, woven, gel, paste, or mesh, wherein said treatment may include spraying, blending, coating, immersion into an impregnation bath, and/or combinations thereof of the said antimicrobial composition of the present disclosure.

In aspects, the method of forming a film, non-woven, woven, gel, paste, or mesh according to the present disclosure, wherein said method may include pre-mixing and/or blending the antimicrobial composition of the present disclosure with the components of the film, non-woven, woven, gel, paste or mesh, prior to the formation of said film, non-woven, woven, gel, paste, or mesh.

In aspects, the invention includes a method of preparing an antimicrobial film, gel, or paste on a surface may include the steps of: a. preparing a mixture of an antimicrobial composition in accordance with the present disclosure; b. optionally, adding at least one solvent and/or fluid to the mixture to form an intermediate mixture; c. applying the mixture and/or the intermediate mixture to the surface, and; d. curing, gelling, cooling, heating, radiating and/or drying the mixture obtained from step c, thereby obtaining an antimicrobial film and/or layer on the surface.

In aspects, the method is a method of preparing the antimicrobial film, gel, or paste on a surface according to the present disclosure, wherein the surface may be a medical device and/or a mammalian tissue. The medical device may be a catheter, a fixation tape, a non-absorbent wound dressing, an absorbent wound dressing, an adhesive, a needle, a tube, a surgical instrument, a tape, an implant, a mask, a scaffold, an ostomy appliance, a collection bag, and combinations thereof.

In another aspect, the invention includes an antimicrobial foam or sponge comprising at least one antimicrobial agent selected from: natural polypeptides, N-acylamino acid esters and/or their salts, esters of glycerol and saturated and/or unsaturated fatty acids (C₆-C₂₀), saturated and/or unsaturated alcohols with C₆-C₂₀ carbon atoms, and combinations thereof; wherein the antimicrobial agent is covalently, ionically, and/or physically bound in the foam or sponge. The foam or sponge may be used in medical applications such as managing external and internal wounds, surgical sites, topical cleaning, and the like.

In aspects, the antimicrobial foam or sponge composition can be based on polymers selected from: silicone and/or its copolymers, polyurethane and/or its copolymers, collagen and/or its derivatives, gelatin and/or its derivatives, cellulose and/or its derivatives and copolymers, polysaccharides and/or their derivatives and copolymers, chitosan and/or its derivatives and copolymers, polyacrylic acid and/or its copolymers and salts, and polyvinyl alcohol and/or its copolymers.

In certain aspects, the antimicrobial composition is an antimicrobial polyurethane foam comprising a reaction product of a polyisocyanate component and a polyol component, and an antimicrobial agent, wherein the antimicrobial agent comprises N^{α}-lauroyl-arginine ester or a salt thereof (for example, N^{α}-lauroyl-arginine ethyl ester or a salt thereof).

In additional aspects, the antimicrobial composition is an antimicrobial polyvinyl alcohol foam wherein the foam comprises N^{α}-lauroyl-arginine ethyl ester or a salt thereof. In certain aspects, the antimicrobial polyvinyl alcohol foam with the antimicrobial agents described herein can be prepared by processes known to those skilled in the art. A suitable method is, for example, frothing the polyvinyl alcohol solution with the antimicrobial agent followed by crosslinking the foam, and drying to yield a foam structure with the antimicrobial agent incorporated within the foam structure. An example of foam manufacturing process is described in U.S. Pat. No. 5,071,648, the contents of which are incorporated by reference herein, which teaches a process for making antimicrobial absorbent materials based on acetalized PVA sponge comprising disinfectant dyes to PVA matrices.

In aspects, the antimicrobial foam or sponge composition according to the present disclosure can further include at least one additional antimicrobial agent with synergistic and/or enhanced antimicrobial activity according to the present disclosure.

In aspects, the antimicrobial foam or sponge composition according to the present disclosure, can further include solvents, hydrophilic additives, pH-buffering agents, stabilizing agents, surfactants, antibiotics, wound healing agents, hormones, growth factors, and combinations thereof.

In another aspect, the invention includes a process for producing a foam or sponge described herein, wherein said process may comprise treating said foam or sponge with a powder, solution, dispersion, emulsion, and/or suspension of said antimicrobial agent.

In aspects, the process is a process for producing a foam or sponge, wherein said treatment may comprise spraying, blending, coating, immersion into an impregnation bath, and/or combinations thereof of the said antimicrobial agent.

In aspects, the process for producing a foam or sponge comprises pre-mixing and/or blending the antimicrobial agent with the polymer prior to the formation of said foam or sponge.

In certain embodiments, the invention is a method for preparing an antimicrobial polyurethane foam, wherein the method comprises reacting a polyisocyanate component and a polyol component in the presence of N^{α}-lauroyl-arginine ester or a salt thereof. In certain aspects, the N^{α}-lauroyl-L-arginine ester or a salt thereof is N^{α}-lauroyl-arginine ethyl ester or a salt thereof.

In another aspect, an antimicrobial composition comprising at least one or more antimicrobial agent selected from: natural polypeptides, N-acylamino acid esters and/or their salts, esters of glycerol and saturated and/or unsaturated fatty acids (C6-C20), saturated and/or unsaturated alcohols with C6-C20 carbon atoms, and combinations thereof; wherein the antimicrobial agent is present in an amount 0.5-90.0 wt %, 5.0-80.0 wt %, or 10.0-70.0 wt %.

In aspects, the antimicrobial composition may be present in the form selected from liquids, gels, creams, foams, lotions, paste, powder, aerosols, and combinations thereof.

In aspects, the antimicrobial composition can further include a chelating agent, present in an amount 0.01-10 wt %, 0.05-5.0 wt %, or 0.1-3.0 wt %. The chelating agent may be selected from the group consisting of ethylenediaminetetraacetic acid (EDTA), diethylenetriaminepentaacetic acid, 2-hydroxyethylethylenediaminetriacetic acid, 1,6-diaminohexamethylenetetraacetic acid, 1,2-diaminocyclohexanctetraacetic acid, O,O′-bis(2-aminoethyl)ethyleneglycoltetraacetic acid, 1,3-diaminopropanetetraacatic acid, N,N′-bis(2-hydroxybenzyl) ethylenediamine-N,N′-diacetic acid, ethylenediamine-N,N′-diacelic acid, ethylenediamine-N,N′-dipropionic acid, triethylenetetraaminehexaacetie acid, ethylenediamine-N,N′-bis(methylenephosphonic acid), iminodiacetic acid, N,N-bis(2-hydroxyethyl)glycine, 1,3-diamino-2-hydroxypropanetetraacetic acid, 1,2-diaminopropanctetraacetic acid, ethylenediaminetetrakis(methylenephosphonic acid), N-(2-hydroxyethyl)iminodiacetic acid, biphosphonates, poly(maleic acid) and its copolymers, poly(maleic anhydride) copolymers, poly(citric acid), polycitrates, polyglutamic acid, polyaspartic acid, poly(succinimide), poly(allylamine) and its copolymers, poly(diallydimethyl ammonium chloride) (polyDADMAC), polyamidoamine (PAMAM) and its copolymers, polyvinylpyrolidone, polystyrenesulfonic acid and/or its salts, poly(styrenesulfonic acid-maleic acid) copolymer and/or its salts, polyacrylic acid and/or its salts, polyacrylic acid copolymers and/or their salts, sulfonated polystyrene and/or its copolymers, and/or their salts, polycitric acid and/or its copolymers, and/or their salts, poly(isobutylene-maleic anhydride) copolymer and/or its salts, polyethyeleneimine and/or its copolymers and/or salts, polyoxazoline and its copolymers and/or salts, hyaluronic acid and its derivatives, chitosan, and combinations thereof.

In aspects, the antimicrobial composition described herein prevents regrowth of biofilm organisms for at least 24 hours after treatment with said antimicrobial composition.

In aspects, the antimicrobial composition according to the present disclosure kills at least about 90% of microbes after exposure to said antimicrobial composition for 24 hours.

In aspects, the antimicrobial composition according to the present disclosure can further include surfactants, hydrophilic additives, pH-buffering agents, solvents, thickening agents, and combinations thereof.

In aspects, the thickening agent is non-ionic, anionic, cationic, amphoteric or combinations thereof, present in an amount of 0.1-50.0 wt %, 0.5-30.0 wt %, or 1.0-20.0 wt %, and may be selected from polyvinylpyrolidone, polystyrenesulfonic acid and/or its salts, polystyrenesulfonic acid-alt-maleic acid and/or its salts, polyalkyleneoxide and/or its copolymers, polyacrylic acid and its copolymers and/or its salts, gums, chitosan, polysaccharides, polypeptides, hydrocolloids, nanoclays, polyacrylamide and its copolymers and/or its salts, and combinations thereof.

In aspects, the antimicrobial composition can be part of a wound cleanser.

In aspects, the invention includes an adhesive composition, wherein the adhesive includes: silicones and/or their copolymers, polyvinylmethyl ether and/or its copolymers, polyacrylates and/or their copolymers, polymethacrylates and/or their copolymers, polyacrylic acid and/or its copolymers, styrenic rubbers, polyvinylpyrrolidone and/or its copolymers, polyvinyl alcohol and/or its copolymers, polyurethanes, polyolefins, and combinations thereof; at least one hydrophilic additive selected from: is selected from citric acid and its salts, glycerols, glycerol esters, monosaccharides, disaccharides, oligosaccharides, polysaccharides, cellulose and its derivatives, hydrocolloids, polyalkylene oxides and their copolymers, polyvinyl alcohol and its copolymers, poly(vinyl pyrrolidone) and is copolymers, poly(vinylmethyl ether) and its copolymers, polymaleic anhydride copolymers, sulfonated polystyrene and its salts and/or copolymers, polyacrylamide and its copolymers, sulfonated polyesters, polyacrylic acid and its copolymers, poly(N-isopropyl acrylamide) and its copolymers, polydimethlyamino methacrylate and its copolymers, gelatin, chitosan, hyaluronic acid, polyamides, polypeptides, polyvinyl amine, polyoxazoline and its copolymers, polyphosphazene and its copolymers, surfactants, polyelectrolytes, and combinations thereof.

In another aspect of the adhesive composition, the hydrophilic additive according to the present disclosure may be a liquid or solution. This may be suitable to lower the overall stiffness of the adhesive and also to deliver any active, if required, which may be dispersed or dissolved in the liquid phase of the adhesive.

In further aspects, wherein the polymer of the adhesive composition can be present in the range of 5 wt % to 99 wt %, 20 wt % to 90 wt %, 30 wt % to 85 wt % or the like. In further aspects, the hydrophilic component may be present in an amount less than 95 wt %, less than 70 wt %, less than 60 wt % or the like.

In aspects, the adhesive composition comprises a surfactant, wherein the surfactant is ionic, non-ionic, and/or amphoteric, and combinations thereof.

In another aspect, the invention includes a wound dressing including a substrate, at least one adhesive to adhere to the wound and/or skin, wherein the adhesive may be according to the present disclosure. Further, the substrate may be selected from polymer film, non-woven, woven fabric, mesh, foams, and combinations thereof.

In another aspect, the invention includes an antimicrobial wound cleanser including at least one or more antimicrobial agent selected from: natural polypeptides, N-acylamino acid esters and/or their salts, esters of glycerol and saturated and/or unsaturated fatty acids (C6-C20), saturated and/or unsaturated alcohols with C6-C20 carbon atoms, and combinations thereof; and at least one surfactant. The antimicrobial agent may be present in an amount 0.5-30.0 wt %, 1.0-20.0 wt %, or 2.0-15.0 wt % of the total composition. Further, the surfactant may be ionic, non-ionic, amphoteric, neutral surfactant, and combinations thereof. The surfactant may be present in an amount less than 20 wt %, less than 15 wt %, or less than 5 wt % of the total composition.

In yet an additional aspect, the invention includes an antimicrobial tissue substitute or scaffold comprising at least one tissue substitute material and at least one antimicrobial agent. In certain aspects, the tissue substitute or scaffold is a skin substitute or scaffold and can include at least one skin substitute material and at least one antimicrobial agent. In certain embodiments, the antimicrobial is an N^{α}-lauroyl-arginine ester or a salt thereof.

In yet additional embodiments, the invention is directed to a method of treating a wound in a subject in need thereof, wherein the wound is at risk for infection, comprising treating the wound with a composition comprising an antimicrobial amount of N^{α}-lauroyl-arginine ester or a salt thereof (including, for example, N^{α}-lauroyl-arginine ethyl ester or a salt thereof).

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIGS. 1A and 1B are bar graphs showing the number of colony forming units (CFUs) of P. aeruginosa and MRSA in log scale for the foam compositions: Mepilex Ag, Kendall AMD, 0.5% Comp A, 1.0% Comp A, 3.75% Comp C, 0.5% Comp B and 1.0% Comp B over 0, 24, 77 and 168 hours.

### DETAILED DESCRIPTION OF THE INVENTION

In medical applications, often medical devices are held on to the patient's body using skin adhesives. Such adhesives are expected to maintain adhesion during use of the device and also remove comfortably when no longer in use. In addition, reducing bio-burden and minimizing risk of infection are important requirements for better patient care and for caregivers. Adhesive and/or antimicrobial compositions play a significant role in medical applications. An effective antimicrobial composition, such as a composition that inhibits growth and proliferation of biofilm embedded microorganisms, can be used in a wide variety of applications. Such an antimicrobial composition can either be used on its own, incorporated into a medical device, or articles as a component or coating, or incorporated onto a surface desirable to be free of microbes or to have reduced bio-burden. The present disclosure provides antimicrobial compositions suitable for medical applications, especially those devices in direct contact with healthy and or denuded skin, wound, surgical incision, tissue, and the like, including antimicrobial agents and compositions that are not cytotoxic, but are effective against bacteria, yeast, and other microbes. The disclosure also describes silicone gel adhesive compositions suitable for medical applications including, but not limited, wound dressings and for holding a medical device to a patient's body.

As used herein, the words “a” and “an” are meant to include one or more unless otherwise specified. For example, the term “an agent” encompasses both a single agent and a combination of two or more agents.

The term “antimicrobial” or “antimicrobial agent” refers to an agent or compound or a composition that kills, inhibits, reduces and/or stops the growth of microorganisms, including, but not limited to, bacteria, virus, fungi, and yeasts.

The term “adhesive” includes to monomers, oligomers, polymers, and combinations thereof that may be used to bond at least two surfaces together temporarily or permanently, and/or may be used to bond to a surface. The term adhesive may also include monomers, oligomers, polymers, and combinations thereof in solution, hydrogel, suspension, and/or emulsion form, which upon drying, curing, or polymerization, may form an adhesive. The adhesive according to the present disclosure may be tacky to touch such as pressure sensitive adhesive. Adhesives described herein also include gel adhesives. The term “skin adhesive” refers to the adhesive described above and is suitable for use on mammalian skin, for example, on human skin. Further the term “adhesive” can also refer to a composition that can temporarily or permanently adhere and/or bond to a surface or between surfaces.

The term “chronic wound” refers to a wound that fails to progress through an orderly and timely sequence of repair or a wound that does not respond to treatment and/or the demands of treatment. Many wounds that are first considered to be acute wounds ultimately become chronic wounds due to factors still not well understood. One significant factor is the transition of planktonic bacteria within the wound to form a biofilm. In the context of wound treatment, “biofilm disruption” or “inhibition of biofilm reconstitution” refers to biofilm clearance from a chronic or acute wound, or to inhibit reconstitution of a biofilm mass from remnants remaining after debridement and thereby promote healing of a wound.

The term “biofilm” refers to a structured community of microorganisms enclosed in a self-produced extracellular polymeric matrix, and attached to a biotic or abiotic surface.

“Treating” or “treatment” includes preventing or delaying the onset of the symptoms, complications, or biochemical indicia of a disease, infection, condition, wound, or disorder, and/or alleviating or ameliorating the symptoms of, alleviating or ameliorating complications related to the care of, or arresting or inhibiting further development of the disease, infection, condition, wound, or disorder. A “subject” is an animal to be treated or in need of treatment. A “patient” is a human subject in need of treatment.

In a first aspect, according to the present disclosure, an antimicrobial adhesive composition includes at least one antimicrobial agent and at least one adhesive.

The antimicrobial agent can, for example, be present in the range of 0.5-90.0 wt %, 5.0-80.0 wt %, or 20.0-70.0 wt %, or the like of the weight of the composition.

The antimicrobial agent can be selected from the group consisting of natural polypeptides, N-acylamino acid esters and/or their salts, esters of glycerol and saturated and/or unsaturated fatty acids (C₆-C₂₀), and saturated and/or unsaturated alcohols with C₆-C₂₀ carbon atoms, and combinations thereof.

The natural polypeptides can be selected from nisin and/or polylysine. Nisin is a polycyclic antibacterial peptide produced by the bacterium Lactococcus lactis used as a food preservative, and has a broad-spectrum activity. Polylysine refers to several types of lysine homopolymers, belonging to the group of cationic polymers: at pH 7, polylysine contains a positively charged hydrophilic amino group. The homopolymers may differ from each other in terms of stereochemistry and link position. The precursor amino acid lysine contains two amino groups, one at the α-carbon and one at the ε-carbon. Polymerization can initiate at either location, resulting in α-polylysine or ε-polylysine. The a-polylysine is a synthetic polymer, which can be composed of either L-lysine or D-lysine resulting in poly-L-lysine (PLL) and poly-D-lysine (PDL); and/or ε-polylysine (ε-poly-L-lysine, EPL). The polylysine may also include modified polylysine such as succinic anhydride modified polylysine. ε-Polylysine is known to have broad-spectrum antibacterial and antifungal activity.

The N-acylamino acid esters and/or their salts can include at least one α-amino acid ester, the α-amino group of which is acylated with a fatty acid, or the corresponding hydrochloride or ammonium salt. The ester of an α-amino acid, such as lysine, arginine or phenylalanine, the α-amino group of which is acylated with a fatty acid, such as lauric acid or stearic acid. The α-amino acid is preferably an L-α-amino acid, as occurs in nature in animal proteins. Preference is given to basic α-amino acids, such as lysine, histidine and arginine. However, hydrophobic α-amino acids can also be used, for example phenylalanine, tyrosine, valine, leucine or isoleucine. The ester of an amino acid generally includes an alkyl ester, including a methyl, ethyl, propyl, isopropyl, butyl, isobutyl, tert-butyl, pentyl, isopentyl, neopentyl, hexyl or isohexyl ester. The fatty acid may include C₆ to C₂₀ (carbon atoms) fatty acid, including lauric acid, myristic acid, palmitic acid and stearic acid. The N-acylated α-amino acid ester is preferably N-lauroyl-L-arginine ethyl ester monohydrochloride (or as also referred to herein as N^{α}-lauroyl arginine ethyl ester hydrochloride) (LAE HCl), N-lauroyl-L-arginine methyl ester monohydrochloride (LAM), or N-lauroyl-L-lysine ethyl ester hydrochloride (LLE). The term “N^{α}-lauroyl-arginine ester or a salt thereof” is meant to include N^{α}-lauroyl-L-arginine esters including, for example, N^{α}-lauroyl-arginine ethyl ester (also referred to as ethyl lauroyl arginate ester, ethyl lauroyl arginate, ethyl lauroyl arginine ester, and ethyl-N^{α}-lauroyl-L-arginate) and salts thereof, such as the hydrochloride salt. In certain aspects of the invention, the preferred N^{α}-lauroyl-arginine ester is N^{α}-lauroyl-arginine ethyl ester or a salt thereof, including, for example, N^{α}-lauroyl-L-arginine ethyl ester hydrochloride.

The esters of glycerol (or glycerol esters) and saturated and/or unsaturated fatty acids (C₆-C₂₀) may include mono, di, and tri-esters. The term “glycerol” includes glycerol, monoglycerol, di-glycerol, tri-glycerol, and poly-glycerol. The saturated fatty acids include, but is not limited to, Caprylic acid, Capric acid, Lauric acid, Myristic acid, Palmitic acid, Stearic acid, and Arachidic acid. The unsaturated fatty acids may include: Myristoleic acid, Palmitoleic acid, Sapienic acid, Oleic acid, Elaidic acid, Vaccenic acid, Linoleic acid, Linoelaidic acid, α-Linolenic acid, Arachidonic acid, and Eicosapentaenoic acid.

The saturated and/or unsaturated alcohols with C₆-C₂₀ carbon atoms include, but are not limited to, hexanol, heptanol, octanol, nonanol, decanol, undecanol, dodecanol, tridecanol, tetradecanol, pentadecanol, hexadecanol, heptadecanol, octadecanol, nonadecanol, eicosanol, phytol, oleyl alcohol, palmitoleyl alcohol, and myristoleyl alcohol.

The antimicrobial adhesive composition can optionally include two or more additional antimicrobial agents including those described herein. For example, the combination of ε-polylysine and N^{α}-lauroyl-L-arginine ethyl ester hydrochloride can be used.

In certain additional aspects, the antimicrobial adhesive composition does not include silver and salts thereof (for example, silver sulfadiazine), chlorohexidine gluconate (CHG), polyhexamethylenebiguanide (PHMB), iodine, hyperchlorous acid and/or octenidine dihydrochloride.

In certain aspects, the adhesive of the antimicrobial adhesive composition can be present at 10.0-90.0 wt %, or 20.0-80.0 wt %, or 40.0-70.0 wt %, or the like of the weight of the composition.

The adhesive of the antimicrobial adhesive composition can, for example, be selected from the group selected from the group consisting of silicones and/or their copolymers, polyvinylmethyl ether and/or its copolymers, polyacrylates and/or their copolymers, polyacrylic acid and/or its copolymers, styrenic rubbers, polyvinylpyrrolidone and/or its copolymers, polyvinyl alcohol and/or its copolymers, polyurethanes, and polyolefins, and combinations thereof.

The silicone adhesive comprises at least one alkenyl- and/or alkynyl-substituted polysiloxane, at least one polysiloxane comprising silicon-bonded hydrogen atoms, and at least one hydrosilylation catalyst and/or a peroxide catalyst. The silicone adhesive comprises at least one alkenyl- and/or alkynyl-substituted polysiloxane covalently crosslinked to the at least one polysiloxane comprising silicon-bonded hydrogen atoms, thereby forming an adhesive or cured gel adhesive. Non-limiting examples of such silicones include Soft Skin Adhesives (SSA) from Dow Corning, such as 7-9900, 7-9800; Silpuran 2130, Silpuran 2100 from Wacker Chemie; Silopren HC2-2022, HC2-2021 from Bluestar Silicones. These compositions are sold as two parts (Part A and B). The two parts of mixed at a specific, for example, Part A: Part B of 1:1, or ratios other than 1:1, and then allowed to set or cure at room temperature or at a higher temperature to form the silicone gel adhesive that is tacky to touch. For addition-cure systems as disclosed above, the crosslinker is typically in the Part B, so a higher amount of Part B may result in a less tacky and/or stiffer adhesive. The terms “crosslinked,” “cross-linked,” and “cured” are used interchangeably to refer to a polymer network that is formed by chemical crosslinking of the polymer chains with chemical moieties with functionality greater than 2. The above terms can also refer to physically crosslinked polymer network, wherein the network comprises of glassy polymer chain segments.

The silicone copolymers may include copolymers of polydimethylsiloxane and polyethers, non-limiting examples include Dow Corning Toray FZ 2233 and Momentive's Silwet 8500; poly-ether-siloxane copolymer networks, cyclopentasiloxane-alkyl cetearyl dimethicone copolymer networks (Momentive's Velvesil 125), vinyldimethyl/trimethylsiloxysilicate stearyl dimethicone crosspolymer, silicone acrylate (DOW CORNING® FA 4001 CM) and the like. An adhesive composition including such silicone copolymers may be combined or mixed with the silicone gel adhesives of the present disclosure.

The silicone adhesive may include at least one polyorganosiloxane such as polydimethylsiloxane, and at least one silicate resin. Such adhesives are pressure sensitive adhesives (PSA), and non-limiting examples of such adhesives are DOW CORNING® MD7-4502 Silicone, DOW CORNING® MD7-4602 Silicone, DOW CORNING® BIO-PSA 7-430X Silicone Adhesive, DOW CORNING® BIO-PSA 7-420X Silicone Adhesive, DOW CORNING® BIO-PSA 7-410X Silicone Adhesive, DOW CORNING® BIO-PSA 7-460X Silicone Adhesive, DOW CORNING® BIO-PSA 7-450X Silicone Adhesive, DOW CORNING® BIO-PSA 7-440X Silicone Adhesive, DOW CORNING® BIO-PSA Hot Melt Adhesive, and the like. The adhesives are typically provided as a solution in organic solvents. Such solutions are coated on a carrier substrate such as films, and heat dried above the boiling point of the solvent(s) to form the adhesive. These silicone adhesives may also be crosslinked to form a cured pressure sensitive adhesive. The curing agents may include organic peroxides, silanes, metallic acetylacetonates, and others that may readily form free radicals when heated up to a certain temperature.

Further, the silicone adhesive may include at least one hydroxyl-terminated polyorganosiloxane, at least one silane, and at least one condensation cure catalyst. Such adhesives are considered to be one-component or two-component RTV (room temperature vulcanizate), which cure via condensation cure. Typically, such adhesives cure in the presence of moisture to yield a rubbery adhesive. Non-limiting examples of such adhesives are Applied Silicone Implant Grade RTV Silicone Adhesives PN 40064 and PN 40076. Such compositions may be rendered tacky to touch by addition of tackifiers such as silicate resins (MQ resins), silicone oils, and/or by blending with silicone PSAs described above. Non-limiting examples of hydroxyl-terminated polysiloxane may include DMS-S12, DMS-S14, DMS-S15, DMS-S21, DMS-S27, DMS-S31, DMS-S32, from Gelest Inc. The condensation catalysts may include be tin-based catalyst such as dibutyl tin laurate, others such as zinc, zirconium, aluminum, and/or titanium-based, combinations thereof and the like. Other catalysts may include those taught by U.S. Pat. App. Pub. No. 2011/0021684 A1, the contents of which are expressly incorporated by reference herein. Other suitable silicones can include those compositions as taught by U.S. Pat. App. Pub. No. 2012/0219517 A1, U.S. Pat. No 6,512,072 B1, and versions of compositions as described in U.S. Pat. No. 6,512,072 B1 without the use of solvents; the contents of each of which are expressly incorporated by reference herein.

Further, the silicone adhesive may include at least one copolymer of 3-[tris(trimethylsilyloxy)silyl]propyl methacrylate (TRIS) and at least one acrylate, wherein the acrylate is selected from n-butyl acrylate, t-butyl acrylate, octyl and/or iso-octyl acrylate, and/or ethylhexyl acrylate. Non-limiting example of such adhesive is 3M™ CAVILON™ No Sting Barrier Film (3M Corporation). Such compositions may be rendered tacky to touch by addition of tackifiers such as silicate resins (MQ resins), or by blending with other PSAs. Further, one could obtain a tacky adhesive by changing the ratio of the comonomer to TRIS in the reaction mixture during copolymerization.

The term “polysiloxane” can refer to polydimethylsiloxane, polydimethylsiloxane with functional groups including hydroxyl, vinyl, acrylate, alkoxy, sulfonate, hydride, polydimethylsiloxane with at least one branch of polyalkyleneoxide, copolymers of polydimethylsiloxane, polydimethylsiloxane with hydrophilic groups such as sulfonic acid and salts, and combinations thereof or the like.

The silicone according to the present disclosure can include at least one alkenyl- and/or alkynyl-substituted polydiorganosiloxane and the at least one polysiloxane comprising silicon-bonded hydrogen atoms may have hydrogen or various hydrocarbon substituents, such as saturated or unsaturated, branched or linear hydrocarbon chains. The polysiloxanes according to the present disclosure may also have polar groups such as sulfonate, amino, quaternary ammonium, polyaklyleneoxide, and other hydrophilic moieties attached to the silicon in the chain. In accordance with the present disclosure, the said organic substituents on the diorganosiloxane or polysiloxane may comprise methyl, ethyl, propyl, butyl, vinyl, allyl, and/or aryl, and combinations of these. The term “alkenyl- and/or alkynyl-substituted polysiloxane” is to be understood as comprising polydiorganosiloxanes substituted with groups comprising saturated and at least one unsaturated carbon-carbon bonds, which could be carbon-carbon double bonds and/or carbon-carbon triple bonds, and combinations thereof. Further, the term “cross-linked,” “crosslinking,” “cured,” or “curing” shall be understood to relate to the cross-link reaction or bond formation that can be created between alkenyl and/ alkynyl moieties (i.e. unsaturations) of at least one polysiloxane and the silicon-hydrogen (Si—H) moiety of a second polysiloxane. Additionally, the term “polysiloxane,” “siloxane,” or “silicone,” shall be understood to pertain to all types of polysiloxanes, for instance, polydiorganosiloxanes, etc., and within the context of the present disclosure, these terms are used interchangeably. Finally, the process feature of “mixing” shall be understood to relate to mixing in any order the components in the mixture, and can include dissolving the components in a solvent, if required, even though it may not be specifically pointed out. Optionally, the polysiloxane may also include a non-reactive polydiorganosiloxane, such as silicone fluids.

The silicone adhesive of the present disclosure may further include silicone and/or silicate resins. Silicone resins may be included to increase the adhesion of the adhesive to skin or any substrate or surface. They are also referred to as tackifiers. Silicone resins are silicone materials formed by branched, cage-like oligosiloxanes with the general formula of RₙSiXₘO_{y}, where R is a non-reactive substituent, usually methyl (Me) or phenyl (Ph), and X is a functional group H, OH, vinyl, or O—R. These groups are further condensed in many applications, to give highly crosslinked, polysiloxane networks. Typical siloxane resins are MQ resins. MQ resins are three-dimensional network of M type and Q type silicon-oxygen structure. Non-limiting examples of commercially available MQ resins are MQ-RESIN POWDER 803 TF from Wacker Chemical Corporation; VQM-135, VQM-146, HQM-105, HQM-107, SQO-299, and SQD-255 from Gelest Inc., Prosil 9932, MQOH-7 from SiVance, LLC. The resins could have specific functionality such as hydroxyl, vinyl, hydride, and the like. In further aspects, other silicone resins such as silsesquioxanes may also be included.

In certain embodiments, the silicone adhesives described herein do not include a silicate resin.

In aspects, the adhesives according to the present disclosure may include polyvinylmethyl ether and/or its copolymers, polyacrylates and/or their copolymers, polyacrylic acid and/or its copolymers, styrenic rubbers, polyvinylpyrrolidone and/or its copolymers, polyvinyl alcohol and/or its copolymers, polyurethanes, polyolefins may also be suitable according to the present disclosure. The polyvinylmethyl ether copolymers include those commercially available under the tradename GANTREZ™ (from Ashland Inc.). Polyacrylates and/or their copolymers, include polyacrylates, poly(meth)acrylates and/or their copolymers. Non-limiting examples of polyacrylate adhesives are available from 3M, for example Medical Permanent Adhesives, P1500 and P1510; from Henkel under tradenames, Durotak and Gelva GMS. Polyacrylic acid and/or methacrylic acid and/or their copolymers, may also be generally referred to as polyacrylates or polymethacrylates. These adhesives are typically a copolymer of different acrylic and/or methacrylic monomers. Such polymers are sold under the tradename, Carbopol (acrylic acid copolymers), Eudragit (methacrylic acid copolymers; registered trademark of Evonik Industries). These adhesives may include plasticizers such as glycerol, alkyl citrates, glycerol esters, adipates, phthalates, polyalkylene oxides, etc. Polyacrylate adhesives may further comprise tackifiers to optimize the rheology of the adhesive and to adjust adhesion and tack properties. The adhesives may be presented as hotmelts or solvent borne. They may also be chemically or physically crosslinked. Adhesives based on styrenic rubbers, comprise the styrenic rubber, tackifiers, plasticizers, etc. The polyolefin adhesives are based on polyisoprene, polyisobutylene, polyethylene, polyethylene-propylene, etc. An example of polyisobutylene adhesive may be DURO-TAK 87-6908 from Henkel North America. Polyurethane adhesives disclosed in accordance with the present disclosure herein include, but are not limited to: those methods, approaches, devices described in: U.S. Pat. No. 6,518,359 B1, the contents of which are herein incorporated by reference.

In certain embodiments, the adhesive of the antimicrobial adhesive composition is an acrylic adhesive, for example, comprising a polyacrylate and/or its copolymers and N^{α}-lauroyl-L-arginine ethyl ester. In certain aspects, the invention encompasses an antimicrobial adhesive comprising at least one adhesive based on polyacrylate or a copolymer thereof and an antimicrobial agent, for example, N^{α}-lauroyl-L-arginine ester or a salt thereof. In yet further aspects, the adhesive composition reduces the number of colony forming units (CFUs) of microbes by at least one log order after about 24 hours of treatment. In certain aspects, the polyacrylate adhesive is present in amount from about 75% to about 95% by weight of the composition, and the antimicrobial agent is present in an amount from about 0.5% to about 10% by weight of the adhesive composition. Antimicrobial compositions based on acrylic adhesives that are solvent-based, can be prepared by adding the antimicrobial agents to the solution, following by coating or applying the mixture on to a surface, followed by drying the surface at room temperature or higher temperature. In cases where the adhesive is a hotmelt, the adhesive can be melted to a flowable temperature, followed by adding the components, mixing, and applying the hotmelt mixture on to a surface. The mixing can be accomplished in shear mixers, such as a Brabender mixer. The surface can, for example, include biological tissue, skin, film, foam, non-woven material, woven material, fabric, sheet, rubber, fibers, mesh, plastic, and combinations thereof.

In certain embodiments, the adhesive of the antimicrobial adhesive composition is a polyurethane adhesive, for example, comprising a polyurethane and N^{α}-lauroyl-L-arginine ethyl ester. The polyurethane adhesive can, for example, be prepared by mixing a polyisocyanate component and a polyol component, and coating the mixture on a suitable substrate such as a film, woven fabric, non-woven fabric, release liners, mesh, fiber, and the like. Optionally, other components can be added, such as a solvent, water, surfactants, chain extenders, and the like. Polyurethane adhesive compositions are described, for example, in U.S. Pat. No. 6,518,359 and U.S. Pat. No. 5,591,820; the contents of each of which are incorporated by reference herein.

The adhesives of the antimicrobial adhesive composition can be hydrophilic, hydrophobic, amphiphilic, and/or ionic in nature. This can be achieved by selecting adhesives with polymers that are hydrophilic, hydrophobic, amphiphilic, and/or ionic, or by formulating with appropriate components and/or additives that render the adhesive formulation hydrophilic, hydrophobic, amphiphilic, and/or ionic.

In certain embodiments, the invention is directed to an antimicrobial adhesive composition comprising:
a. a silicone gel adhesive in an amount of about 75 to about 95% by weight, wherein the silicone gel adhesive is prepared via hydrosilylation in the presence of a platinum catalyst;
b. a N^{α}-lauroyl-arginine ester or a salt thereof in an amount of about 0.5 to about 10% by weight; and
c. a non-ionic additive in an amount of about 0.5 to about 10% by weight.

In some embodiments, the non-ionic additive is a non-ionic hydrocolloid. In yet additional aspects, the non-ionic additive is a cellulose. In certain aspects, the non-ionic additive is selected from the group consisting of hydroxyethyl cellulose, hydroxypropyl cellulose, methyl cellulose, carboxymethylcellulose, maltodextrin, dextran, xanthan gum, guar gum, pectin, beta-glucans, rice protein, oat protein, potato protein, and polylysine. The N^{α}-lauroyl-arginine ester or a salt thereof is preferably N^{α}-lauroyl-arginine ethyl ester or a salt thereof.

As described above, N^{α}-lauroyl-arginine ethyl ester (LAE) is an amide-ester of lauric acid and arginine, wherein the acid group in arginine is esterified with ethyl group. N^{α}-lauroyl-arginine ethyl ester hydrochloride has been described as an antimicrobial agent and is used in food and meat preservation. LAE is cationic and is sensitive to pH and highly anionic or highly polar additives. LAE also contains free-amino groups which, when added during the preparation of a silicone gel adhesive, can potentially have a negative effect on the platinum catalyst used a silicone gel formulation. Indeed, as described in the Examples below, when LAE is added to a liquid silicone gel adhesive composition and cured to form an adhesive, the resulting adhesive is under-cured and has little or no cohesive strength. Surprisingly, it has been found that the addition of a non-ionic additive to the silicone gel with LAE overcomes the cure issue, and the resulting adhesive is cohesively stronger and displays antimicrobial properties.

The antimicrobial adhesive composition can optionally further comprise glycerol, a glycerol ester or a glycerol ether; for example, the composition can further comprise, 0.01 to about 10% by weight of a glycerol, glyceryl alkyl ether or glyceryl alkyl ester. In certain embodiments, the non-ionic additive is hydroxyethyl cellulose or hydroxypropyl cellulose. In yet additional aspects, the non-ionic additive is hydroxyethyl cellulose. In yet additional aspects, the non-ionic additive is polylysine. In some embodiments, the silicone gel adhesive is an amount of about 80 to about 90% by weight, the N^{α}-lauroyl-arginine ethyl ester or a salt thereof is in an amount of about 1 to about 5% by weight, and the hydroxyethyl cellulose is present in an amount of about 2 to about 7% by weight; and optionally further comprising glycerol in an amount of about 0.01 to about 10% by weight. In yet additional aspects, the silicone gel adhesive is in an amount of about 85% by weight, the N^{α}-lauroyl-arginine ethyl ester or a salt thereof is in an amount of about 2% by weight, and the hydroxyethyl cellulose is present in an amount of about 5% by weight, and wherein the composition optionally further comprises glycerol in an amount of about 8% by weight. In further aspects, the non-ionic additive is maltodextrin; for example, the silicone gel adhesive is an amount of about 90 to about 95% by weight, the N^{α}-lauroyl-arginine ester or a salt thereof is in an amount of about 1 to about 5% by weight, and the maltodextrin is present in an amount of about 1 to about 5% by weight. In additional aspects, the silicone gel adhesive is present in an amount of about 95% by weight, the N^{α}-lauroyl-arginine ester or a salt thereof is present in an amount of about 2.5% by weight, and the maltodextrin is present in an amount of about 2.5% by weight. The adhesive composition can be prepared by crosslinking an alkenyl and/or alkynyl-substituted polydiorganosiloxane with a polysiloxane comprising silicon-bonded hydrogen atoms, wherein the crosslinking is conducted in the presence of the platinum catalyst, the N^{α}-lauroyl-arginine ester or a salt thereof and the non-ionic cellulose.

The antimicrobial adhesive compositions herein can be optimized for the extent of adhesiveness versus the antimicrobial effect based on the given use of said antimicrobial adhesive composition. For example, the antimicrobial adhesive composition used in articles such as infusion pump or an ostomy appliance, may require higher level of adhesion; in such cases, the adhesive may be present at a higher level. Similarly, the antimicrobial adhesive composition used in articles such as intravenous lines (IV) or in an infection prone area such as a surgical site, may require high level of antimicrobial effect.

In aspects, the antimicrobial composition according to the present disclosure may further comprise additional components such as solvents, wetting agents, process aids, and the like.

In further aspects, the antimicrobial composition may be delivered as a tape, a film, an adhesive, a layer, a non-perforated sheet, a perforated sheet, a foam, a woven material, a non-woven material, a fiber, a porous membrane, a non-porous membrane, and combinations thereof. Such delivery forms may be easily obtained by existing manufacturing methods in the field. The antimicrobial composition may be prepared as a liquid or semi-solid or heat-fusible mass, which is then coated on a substrate such as film, foam, nonwoven, fabric, perforated sheet, membranes, and the like, using roll coaters, sprayers, and other known techniques. The resulting coating can be cooled, heated, dried, or simply processed to final shapes as required.

In aspects, the antimicrobial composition of the present disclosure may further include at least one additional antimicrobial agent in addition to those described herein, with synergistic and/or enhanced antimicrobial activity. The use of the term, “synergistic” in the present disclosure refers to a biological effect created from the application of two or more agents to produce a biological effect that is greater than the sum of the biological effects produced by the application of the individual agents. This additional antimicrobial agent may complement the effect of the primary agent, enhance, and/or broaden the spectrum of antimicrobial activity. The additional antimicrobial agent may be selected from curcumin, 2-phenoxyethanol, tea tree oil (Melaleuca oil), natural oils, xylitol and its esters, lactoferrin, chlorhexidine salts, polymeric biguanides, non-polymeric biguanidines, hexetidine and its salts, quaternary ammonium compounds, cetylpyridinium salts, chloramine T, and metals including their oxides and salts, wherein the metal is selected from copper, zinc, and/or silver, and combinations thereof. The amount of such additional antimicrobial agent may be present in an amount to have a synergistic or enhancing effect of the antimicrobial composition. The additional antimicrobial agent may be present in the range of 0.01-60.0 wt %, 0.5-50.0 wt %, or 1.0-40.0 wt %, or the like of the weight of the composition. The silver salts may be selected from silver sulfate, silver sulfite, silver nitrate, silver carbonate, silver phosphate, silver zirconium, and/or organic silver salts, such as silver citrate, silver acetate, silver lactate, and/or combinations or mixtures thereof. The copper salts may include salts of Cu(I) and Cu(II). The zinc salts may include zinc sulfate, gluconate, acetate, and the like.

In certain additional aspects, the antimicrobial adhesive composition does not comprise silver and salts thereof (for example, silver sulfadiazine), chlorohexidine gluconate (CHG), polyhexamethylenebiguanide (PHMB), iodine, hyperchlorous acid and/or octenidine dihydrochloride.

In further aspects, the antimicrobial composition can comprise at least one surfactant. The surfactant may influence the compatibility between the components, processability, and/or the performance of the antimicrobial adhesive. The surfactant may be selected from glycerols, silicone glycerol, silicone-polyether copolymers, polyalkylene oxides, quaternary ammonium salts, polysorbate, fatty acid esters, sugar esters, alkyl sulfates, sulfosuccinates, and combinations thereof. One or more of these surfactants can be used together to obtain the composition. The amount of surfactant in the composition may be present in the range of 0.1-40.0 wt %, 1.0-30.0 wt %, or 2.0-20.0 wt %, or the like of the weight of the composition.

In further aspects, the antimicrobial composition can further comprise at least one hydrophilic additive, wherein the hydrophilic additive is swellable, soluble, dispersible, and/or forms gels in aqueous medium. Further the hydrophilic additive according to the present disclosure may be a liquid or solution. The hydrophilic additive may influence the moisture management or moisture vapor transmission rate (MVTR), the antimicrobial activity, and/or biocompatibility of the antimicrobial adhesive composition. The hydrophilic additive may be selected from citric acid and its salts, glycerols, glycerol esters, monosaccharides, disaccharides, oligosaccharides, polysaccharides, cellulose and its derivatives, hydrocolloids, polyalkylene oxides and their copolymers, polyvinyl alcohol and its copolymers, poly(vinyl pyrrolidone) and is copolymers, poly(vinylmethyl ether) and its copolymers, polymaleic anhydride copolymers, sulfonated polystyrene and its salts and/or copolymers, polyacrylamide and its copolymers, polyN-alkylacrylamide and its copolymers, sulfonated polyesters, polyacrylic acid and its copolymers, poly(N-isopropyl acrylamide) and its copolymers, polydimethlyamino methacrylate and its copolymers, gelatin, chitosan, hyaluronic acid, polyamides, polypeptides, polyvinyl amine, polyoxazoline and its copolymers, polyphosphazene and its copolymers, and combinations thereof. The hydrophilic additive may be present in the range of 1.0-40.0 wt %, 2.0-30.0 wt %, or 5.0-20.0 wt %, or the like of the weight of the composition.

An antimicrobial adhesive composition can comprise an adhesive selected from silicones and/or their copolymers, polyvinylmethyl ether and/or its copolymers, polyacrylates and/or their copolymers, polymethacrylates and/or their copolymers, polyacrylic acid and/or its copolymers, styrenic rubbers, polyvinylpyrrolidone and/or its copolymers, polyvinyl alcohol and/or its copolymers, polyurethanes, polyolefins, and combinations thereof; at least one hydrophilic additive selected from: citric acid and its salts, glycerols, glycerol esters, monosaccharides, disaccharides, oligosaccharides, polysaccharides, cellulose and its derivatives, hydrocolloids, polyalkylene oxides and their copolymers, polyvinyl alcohol and its copolymers, poly(vinyl pyrrolidone) and is copolymers, poly(vinylmethyl ether) and its copolymers, polymaleic anhydride copolymers, sulfonated polystyrene and its salts and/or copolymers, polyacrylamide and its copolymers, sulfonated polyesters, polyacrylic acid and its copolymers, poly(N-isopropyl acrylamide) and its copolymers, polydimethlyamino methacrylate and its copolymers, gelatin, chitosan, hyaluronic acid, polyamides, polypeptides, polyvinyl amine, polyoxazoline and its copolymers, polyphosphazene and its copolymers, surfactants, polyelectrolytes, and combinations thereof.

The polymer can be present in the range of about 5 wt % to 99 wt %, 20 wt % to 90 wt %, 30 wt % to 85 wt % or the like. The polymer contributes to the adhesiveness of the composition by itself or by combination with other components. The amount of polymer may be adjusted according to the level of adhesion required. For example, for low adhesion, lower polymer level may be used. The polymer may be linear, branched or crosslinked molecular structure. For example, the silicone gel adhesive may be considered as crosslinked structure.

In further aspects, the adhesive composition comprises a hydrophilic component, wherein the hydrophilic component may be present in an amount less than 95 wt %, less than 70 wt %, less than 60 wt % or the like.

In order to prepare the silicone adhesive, the unreacted components of the silicone gel adhesive may be combined with the hydrophilic and/or other components prior to curing or crosslinking the gel adhesive.

In order to prepare polyacrylate adhesive or similar adhesives, the adhesive may be dissolved in suitable solvents and the hydrophilic component(s) added to the mixture. The final adhesive may be obtained by drying the mixture at room temperature or at higher temperatures.

The hydrophilic component allows the composition to manage moisture better and improving the moisture vapor transmission rate (MVTR).

The adhesive composition can comprise a hydrophilic component, wherein the hydrophilic component can be at least one surfactant, wherein the surfactant may be ionic, non-ionic, and/or amphoteric, and combinations thereof. Examples of suitable surfactants may include alkyl sulfates, sulfosuccinates, polyethers such as polyethyleneglycol, polyethylene glycol-polypropylene glycol copolymers, phosphonates, fatty acid esters, citric acid esters, sulfonates, and the like.

The adhesive composition can comprise a hydrophilic component, wherein the hydrophilic component may be at least one polyelectrolyte, wherein the polyelectrolyte may be characterized as a polymeric structure with repeating charge moieties. Non-limiting examples may include polyallylamine hydrochloride, poly dimethylaminoethyl methacrylate, and the like.

Moisture vapor transmission rate (MVTR) can be measured using an upright cup method or inverted cup method according to ASTM D3833/D3833M-96(2011) Standard Test Method for Water Vapor Transmission of Pressure-Sensitive Tapes. The test results are reported as grams per square meter per 24 hours. The adhesives described herein can have MVTR values greater than 200 g/m² over 24 hours in an upright cup method measured at room temperature to 38° C., and relative humidity of 50-98%.

In aspects, the adhesive or antimicrobial adhesive composition of the present disclosure can have peel adhesion or strength of 0.1-10.0 N/in, 0.2-8 N/in, or 0.5-6 N/in, against stainless steel tested per ASTM D3330/D3330M-04, method A. The peel test method may be modified or other suitable methods and standards may also be utilized. For example, the stainless steel substrate maybe replaced with polycarbonate substrate, which may be appropriate for softer gel compositions, for example silicone gels.

In some embodiments, the adhesive or antimicrobial adhesive composition has a peel adhesion of adhesive tape to PSTC Stainless Steel is about 5 to about 1000 g/inch, about 10 to about 700 g/inch, or about 15 to about 500 g/inch as measured according to ASTM D3330/D3330M-04, method A.

In certain additional aspects, the antimicrobial adhesive composition leaves little or no residue on the skin. This can, for example, be measured using ASTM D3330/D3330M-04, method A, where the stainless steel plate is examined for adhesive residue or transfer after the tape has been peeled of the plate per the ASTM standard or modifications of the Test method.

An exemplary method of preparing an antimicrobial adhesive composition comprising a silicone gel adhesive and N^{α}-lauroyl-arginine ester or a salt thereof, is a method comprising the steps of:
a. preparing a mixture comprising an alkenyl and/or alkynyl-substituted polydiorganosiloxane, a polydiorganosiloxane comprising silicon-bonded hydrogen atoms, a platinum catalyst, N^{α}-lauroyl-arginine ester or a salt thereof, and a non-ionic additive; and
b. curing the above mixture from (a) on a carrier;
   wherein the carrier a polymer film, non-woven, woven fabric, mesh, foam, gel, and a combination thereof. The non-ionic additive includes, for example, hydroxyethyl cellulose, hydroxypropyl cellulose, methyl cellulose, carboxymethylcellulose, maltodextrin, dextran, xanthan gum, guar gum, pectin, beta-glucans, rice protein, oat protein, potato protein, and polylysine. The non-ionic additive can, for example, be present in the mixture in an amount of about 0.5 to about 10% by weight. The N^{α}-lauroyl-arginine ester or a salt thereof can be present in the mixture in an amount of about 0.5 to about 10% by weight; for example, N^{α}-lauroyl-arginine ethyl ester or a salt thereof can be present in the mixture in an amount of about 0.5 to about 10% by weight.

In addition to the antimicrobial adhesive compositions discussed above, the invention also encompasses certain silicone gel adhesive compositions, such as skin adhesive compositions, that may or may not include an antimicrobial agent. When such silicone gel adhesive compositions include an antimicrobial agent, it is to be understood that these compositions are encompassed within the term “antimicrobial composition” and “antimicrobial adhesive composition.” Moisture management is an important consideration for such skin adhesives. In wound care, skin adhesives are used, for example, to secure wound dressings and adhesive tapes to the body. It is important for such devices, dressings and tapes to stay in place for exudate management and to promote wound healing. Silicone gel adhesives are used in dressings due to their gentle adhesion and non-traumatic removal. Since silicone adhesives are hydrophobic, they have low moisture vapor transmission rate (MVTR), about 150-200 grams per sq meter per 24 hours, which is lower than the normal breathability of skin which is about 500 grams per sq meter per 24 hrs. Due to the differences in MVTR, moisture can collect under the dressing or adhesive which, in turn, can lead to skin maceration and/or result in the dressing falling off. Furthermore, in the presence of exudate, the dressing can fail due to loss of adhesion. Formulating an adhesive to maintain skin adhesion under wet conditions (e.g., wound exudate) and to manage moisture (from perspiration and breathability of skin) is challenging. This is because skin adhesion requires a soft and tacky adhesive, while moisture management requires non-adhesive hydrophilic additives that can stiffen up the adhesive and reduce adhesion. Achieving a balance between tackiness or dry adhesion and MVTR is required to design useful dressings that can stay in place for the intended duration. An ideal wound dressing with silicone gel adhesive, has an MVTR equal to or greater than that of skin (500 grams per square meter per 24 hours) and stays in place for several days in the presence of exudate. Another problem in formulating with silicone gel adhesive is the presence of platinum catalyst, which can be poisoned or negatively impacted by polar additives (cationic, anionic, hydroxyl, acidic groups), amines, sulfur-based compounds, and the like.

The invention thus encompasses hydrophilic silicone gel adhesive compositions that can optionally further contain an antimicrobial agent that displays the balance between dry adhesion (or tackiness) and MVTR. In some embodiments, the invention is directed to a hydrophilic silicone gel adhesive comprising:
a. polydimethylsiloxane in an amount of about 75 to about 95% by weight, wherein the polydimethylsiloxane is crosslinked by hydrosilylation in the presence of a hydrosilylation catalyst;
b. a non-ionic cellulose in an amount of about 1 to about 10% by weight; and
c. a plasticizing agent for the non-ionic cellulose in an amount of about 0.5 to about 20% by weight, wherein the plasticizing agent is selected from the group consisting of glycerol, glyceryl alkyl ether and glyceryl alkyl ester.

The non-ionic cellulose can, for example, be is a non-ionic cellulose ether such hydroxyethyl cellulose and hydroxypropyl cellulose. In certain aspects, the non-ionic cellulose has a viscosity greater than about 500 mPa in a 1% aqueous solution. In yet further aspect, the non-ionic cellulose has a viscosity greater than about 10,000 mPA in a 1% aqueous solution. In yet additional aspects, the non-ionic cellulose has molecular weight such that its viscosity in a 1% aqueous solution is greater than about 1,000 cP, or greater than about 5,000 cP, or greater than about 10,000 cP. The adhesive described herein can, for example, have a moisture vapor transmission rate (MVTR) of greater than or equal to about 500 grams/square meter per 24 hours at 37° C., or at least about 700 grams/square meter per 24 hours at 37° C., for example, as measured using the upright cup method of ASTM E96 with an adhesive thickness that can range from 10 to 250 microns, or 25 to 200 microns, or 25 to 175 microns. In yet additional aspects, the adhesive has an MVTR between about 650 and about 1500 grams/sq m per 24 hours, or between about 700 and 1,000 grams/square meters per 24 hours. In yet additional aspects, the adhesive is characterized by a peel adhesion of adhesive tape to PSTC Stainless Steel is about 10 to about 240 g/inch, for example, as measured according to ASTM D3330/D3330M-04, method A. In yet additional aspects, the adhesive has peel adhesion to PSTC Stainless Steel that is greater than about 5 g/inch, or greater than about 10 g/inch as measured according to ASTM D3330/D3330M-04, method A. The adhesive can optionally further comprise an antimicrobial agent, for example, N^{α}-lauroyl-arginine ester or a salt thereof. The N^{α}-lauroyl-arginine ester or a salt thereof can, for example, be N^{α}-lauroyl-arginine ethyl ester (LAE) or a salt thereof. In yet additional aspects, the invention includes a wound dressing comprising a substrate and the silicone gel adhesive. The substrate can, for example, be a polymer film, non-woven, woven fabric, mesh, foam, gel, and a combination thereof. In certain embodiments, the substrate is a film, for example, a film comprising polyurethane. The invention also includes a method of treating a wound in a subject in need thereof comprising applying the wound dressing to the wound. The adhesive comprising an antimicrobial, for example, N^{α}-lauroyl-arginine ethyl ester, can be used to prevent or treat a biofilm (for example, a biofilm in a wound bed) in a subject in need thereof. The invention further includes a method of securing a medical device to the body or the skin of a subject comprising adhering the medical device to the body or to the skin using the hydrophilic silicone gel adhesive described herein.

In yet additional aspects, the silicone gel adhesive comprises silicone gel adhesive (blend of Part A+Part B) at about 85% by weight of the composition, glycerol at about 10% by weight of the composition, and hydroxyethyl cellulose at about 5% by weight of the composition. The gel adhesive composition can be prepared by mixing the components, coating and curing on polyurethane film at a temperature from about 140 to about 150° C. and protecting the resulting cure adhesive with a release liner.

In aspects, the adhesive and/or antimicrobial adhesive composition described herein may be tacky to touch, when probed by a clean and dry finger. The peel adhesion and/or tackiness of the adhesive composition of the present disclosure may be optimized for the application. When the application for example involves a surgical site, a low adhesion but tacky and gentle adhesive may be required, so that the composition does not cause trauma on removal. On the other hand, when the application involves a wound dressing, moderate adhesion but tacky adhesive may be required for quick stick but gentle on removal. In certain additional aspects, the adhesive composition leaves little or no residue on the skin. It should be noted that tackiness is a measure of the readiness of the adhesive to wet and bond to the surface. This occurs in short time span compared to peel adhesion test, which is a long time span, wherein the interface between the adhesive and the surface it is bonded to, is subjected to a force to separate the two, and the resistance to this separation is a measure of the peel adhesion or strength.

In another aspect, a method of preparing an adhesive or antimicrobial adhesive layer on a surface comprises: i. preparing a mixture of an adhesive composition in accordance with the present disclosure; ii. optionally, adding at least one solvent and/or fluid to the mixture to form an intermediate mixture; iii. applying the mixture and/or the intermediate mixture to the surface to form a layer and; iv. curing, gelling, cooling, heating, radiating and/or drying the layer, thereby obtaining an antimicrobial adhesive layer on the surface. The solvent choice may be dependent on the adhesive chemistry, and if the adhesive (pre-reaction or pre-curing) is a liquid or not. The surface may include a pre-coating of primers, adhesion promoters, or the like to improve adhesion of the composition to the surface.

For example, when silicone gel adhesives are used, the antimicrobial agents and/or other components of the adhesive can be mixed into Part A or Part B, prior to curing, applying the mixture of the two parts plus the antimicrobial agents on to a surface, followed by curing the composition. The surface can, for example, be paper, a polymer film, a rubber, a device, a fabric, a non-woven, and the like.

In certain additional embodiments, the invention includes an antimicrobial adhesive comprising a polyurethane adhesive, wherein the polyurethane adhesive is the reaction product of a polyisocyanate component and a polyol component an antimicrobial agent, for example N^{α}-lauroyl arginine ethyl ester or a salt thereof. The antimicrobial composition comprising a polyurethane adhesive can, for example, be prepared by reacting the polyisocyanate component and the polyol component of the adhesive in the present of the N^{α}-lauroyl arginine ethyl ester or a salt thereof. In certain aspects, the adhesive composition reduces the number of colony forming units (CFUs) of microbes by at least one log order after about 24 hours of treatment.

In another aspect, the invention is a method of delivering an adhesive or an antimicrobial adhesive composition to a wound, wherein the method comprises preparing the composition in accordance with the present disclosure, and applying the preparation to the wound. The composition to be delivered to the wound may include a paste, gel, solution, emulsion, tape, adhesive, hydrogel, and the like.

In another aspect, a method of delivering an antimicrobial composition to a biofilm comprises preparing the antimicrobial composition in accordance with the present disclosure, and applying the preparation to the biofilm. The composition can be delivered to the biofilm before and/or after debridement. The method of delivery can be through a dressing that may be in contact with the wound.

In aspects, the antimicrobial composition described herein can reduce the number of colony forming units (CFUs) of Staphylococcus aureus, Pseudomonas aeruginosa, E. coli, Aspergillus brasiliensis, Methicillin-resistant Staphylococcus aureus (MRSA), C. albicans, and/or aspergillus niger by at least one order of magnitude in 24 hours of exposure. In some aspects, the adhesive composition described herein reduces the number of colony forming units (CFUs) of Staphylococcus aureus and Pseudomonas aeruginosa by at least one order of magnitude after about 24 hours.

In further aspects, the adhesive or antimicrobial adhesive composition of the present disclosure can be applied on a medical device as an antimicrobial layer, wherein the medical device may be a catheter, a fixation tape, a cover dressing, an absorbent dressing, a needle, a tube, a surgical instrument, a tape, an implant, a mask, a scaffold, an ostomy appliance, a collection bag, and combinations thereof.

In another aspect, the wound dressing of the present disclosure may include a skin adhering region, wherein the skin adhering region includes the adhesive or antimicrobial composition described herein. In another aspect, the wound dressing according to the present disclosure may include an absorbent region and a skin adhering region, wherein the absorbent region and/or the skin adhering region comprises the antimicrobial composition in accordance with the present disclosure.

In certain embodiments, the wound dressing comprises a skin adhering region, wherein the skin adhering region comprises the adhesive composition comprising:
a) a silicone gel adhesive in an amount of about 75 to about 95% by weight, wherein the silicone gel adhesive is prepared via hydrosilylation in the presence of a platinum catalyst;
b) a N^{α}-lauroyl-arginine ester or a salt thereof in an amount of about 0.5 to about 10% by weight; and
c) a non-ionic additive selected from the group consisting of hydroxyethyl cellulose, hydroxypropyl cellulose, methyl cellulose, carboxymethylcellulose, maltodextrin, dextran, xanthan gum, guar gum, pectin, beta-glucans, rice protein, oat protein, potato protein, and polylysine, wherein the non-ionic additive is present in an amount of about 0.5 to about 10% by weight.

In an additional aspect, the wound dressing of the present disclosure may include an antimicrobial composition according to the present disclosure, wherein the antimicrobial composition further includes at least one delivery agent, and the composition may include two phases including a continuous phase and a discontinuous phase, wherein the continuous phase may include the adhesive, and the discontinuous phase may include the antimicrobial agent and the delivery agent, wherein the delivery agent breaks down in the wound environment or physiological fluid to release the antimicrobial agent.

The delivery agent can be selected from citric acid and/or its salts, glycerols, glycerol esters, polyalkylene oxides and their copolymers, monosaccharides, oligosaccharides, polysaccharides, polyvinyl alcohol and its copolymers, poly(vinyl pyrrolidone) and is copolymers, poly(vinylmethyl ether) and its copolymers, polymaleic anhydride copolymers, sulfonated polystyrene and its salts and/or copolymers, polyacrylamide and its copolymers, sulfonated polyesters, polyacrylic acid and its copolymers, poly(N-isopropyl acrylamide) and its copolymers, polydimethlyamino methacrylate and its copolymers, gelatin, chitosan, hyaluronic acid, polyamides, polypeptides, polyvinyl amine, polyoxazoline and its copolymers, polyphosphazene and its copolymers, hydrocolloids, and combinations thereof. The delivery agent may be present in the range of 0.5-80.0 wt %, 2.0-60.0 wt %, or 10.0-50.0 wt %, of the weight of the composition.

Further the delivery agent according to the present disclosure may be a liquid or solution. This may be suitable to lower the overall stiffness of the construction and also to deliver the active, which may be dispersed or dissolved in the liquid phase of construction.

The adhesive or antimicrobial composition according to the present disclosure may further include pH-buffering agent(s). Suitable buffers to adjust pH can include but not limited to citrate salts (sodium and potassium), citric acid, phosphates such as sodium dihydrogen phosphate, disodium monophosphate, boric acid, sodium borate, tartrate, phthalate, tris-(hydroxymethyl)aminomethane, succinate, acetate, propionate, maleate salts, other buffers (such as ACES), and combinations thereof. One or more buffers can be added to antimicrobial compositions of the present disclosure in amounts ranging between approximately 0.05 to 10.0 wt %, or 0.1 to 5.0 wt % of the total weight of the composition.

The antimicrobial compositions described herein can be used to treat an infection, a wound, and/or a biofilm. For example, the antimicrobial compositions described herein can be used to treat a wound is at risk of infection, including, for example, bacterial infection, viral infection, fungal infection and/or parasitic infection. In certain embodiments, the number of colony forming units (CFUs) of Staphylococcus aureus, Pseudomonas aeruginosa, E. coli, Aspergillus brasiliensis, Methicillin-resistant Staphylococcus aureus (MRSA), C. albicans, and/or aspergillus niger is reduced by at least one order of magnitude after about 24 hours of treatment. In some embodiments, the number of colony forming units (CFUs) of Staphylococcus aureus and Pseudomonas aeruginosa is reduced by at least one order of magnitude after about 24 hours of treatment.

In an additional aspects, the invention encompasses an antimicrobial film, non-woven, woven, gel, paste, or mesh including an antimicrobial composition wherein the antimicrobial composition includes at least one antimicrobial agent according to the present disclosure and at least one polymer and/or oligomer, wherein the polymer and/or oligomer may be selected from: silicones and/or their copolymers, polyvinylmethyl ether and/or its copolymers, polyacrylates and/or their copolymers, polymethacrylates and/or their copolymers, polyacrylic acid and/or its copolymers, and/or its salts, styrenic rubbers, polyvinylpyrrolidone and/or its copolymers, polyvinyl alcohol and/or its copolymers, polyurethanes, polycarbonates, polyamides and/or their copolymers, polyesters and/or their copolymers, polyolefins, polyvinyl chloride, polyethersulfone, polyether ether ketone (PEEK), polyalkylene oxides, polysaccharides, chitosan, polypeptides, and combinations thereof.

In aspects, the antimicrobial film, non-woven, woven, gel, paste, or mesh according to the present disclosure may inhibit the growth of Staphylococcus aureus and/or Pseudomonas aeruginosa by at least one order of magnitude in 24 hours according to the test disclosed in the present disclosure.

In aspects, the antimicrobial film, non-woven, woven, gel, paste, or mesh can inhibit the growth of Staphylococcus aureus and Pseudomonas aeruginosa in a zone of inhibition (ZOI) test, wherein the ZOI is at least equal to the size of said film, non-woven, woven, gel, paste, or mesh exposed to the agar plate, when tested according to the test disclosed in the present disclosure.

In aspects, the antimicrobial film, non-woven, woven, gel, paste, or mesh comprises a polymer, wherein the polymer and/or oligomer may be present at 10.0-90.0 wt %, or 20.0-70.0 wt %, or 40.0-60.0 wt % of the weight of the composition.

The antimicrobial film, non-woven, woven, gel, paste, or mesh can comprise a polymer, wherein the polymer and/or oligomer includes silicones, wherein the silicones are according to the present disclosure.

In aspects, the antimicrobial film, non-woven, woven, gel, paste, or mesh comprises the antimicrobial agent in the range of 0.5-90.0 wt %, 5.0-80.0 wt %, or 10.0-70.0 wt %, of the weight of the composition.

In certain additional aspects, the invention is directed to an antimicrobial wound gel comprising:
a. N^{α}-lauroyl-arginine ester or a salt thereof in an amount between about 0.05 to about 3% by weight of the composition; and
b. a non-ionic thickener selected from the group consisting of hydroxyethylcellulose, hydroxypropyl cellulose, methyl cellulose, and polyethylene oxide in an amount between about 0.5 to about 5% by weight of the composition; wherein the wound gel is an aqueous gel with a viscosity greater than 1,000 centipoise.

In certain aspects, the N^{α}-lauroyl-arginine ester or a salt thereof is N^{α}-lauroyl-arginine ethyl ester or a salt thereof. The composition can optionally further comprise polyethylene glycol and/or a buffer. In some embodiments, the non-ionic thickener is selected from the group consisting of hydroxyethyl cellulose, hydroxypropyl cellulose, and methyl cellulose. In certain additional aspects, the non-ionic thickener is hydroxyethyl cellulose or hydroxypropyl cellulose. In yet further aspects, the compositions comprise PEG 8 in an amount of about 5%, hydroxyethyl cellulose in an amount of about 2% and N^{α}-lauroyl-arginine ethyl ester in an amount of about 0.7%. In additional aspects, additional ingredients suitable for a wound gel can be added, for example, glycerol, iodine, salts, other thickeners such as polyacrylates, starches, celluloses, gelatin, polysaccharides, and the like. In yet additional aspects, the composition does not comprise an additional antimicrobial agent selected from the group consisting of silver and salts thereof (for example, silver sulfadiazine), chlorohexidine gluconate (CHG), polyhexamethylenebiguanide (PHMB), iodine, hyperchlorous acid and/or octenidine dihydrochloride. One of the advantages of the gel described herein is that the gel is substantially non-toxic to the skin or is skin-safe. Whether the gel is skin-safe or substantially non-toxic to the skin can, for example, be determined using the ISO 10993 tests for biocompatibility including ISO10993 Part 5 (Cytotoxicity), ISO10993 Part 10 (Skin irritation), and ISO10993 Part 10 (Skin sensitization). In certain aspects, the wound gels described herein have a Grade 3 or below for Reactivity grades for agar and filter diffusion test and direct contact test (ISO10993 Part 5); Erythema and Oedema below an irritation score of 2 or Primary or cumulative irritation score in rabbits of less than 2.0 (ISO10993 Part 10); and/or Magnusson and Kligham scale rating equal to or below 1 (ISO10993 Part 10).

In some embodiments, the invention is a method of treating a wound in a subject in need thereof, wherein the wound is at risk for infection, comprising treating the wound with the wound gel described herein. In certain additional aspects, the method is a method of treating a burn, scar, bacterial infection, viral infection, and/or fungal infection in a subject in need thereof comprising treating the affected area with the wound gel. In certain aspects, the wound is selected from the group consisting of venous stasis ulcers, skin sores, pressure sores, surgical wounds, burns and diabetic foot ulcer. In yet additional embodiments, the wound is a diabetic foot ulcer, skin tear, a pressure ulcer including stage IV.

In another aspect, the invention includes a method of forming an antimicrobial film, non-woven, woven, gel, paste, or mesh according to the present disclosure, wherein the said method may include treating said film, non-woven, woven, gel, paste, or mesh with a powder, solution, dispersion, emulsion, and/or suspension of said antimicrobial composition according to the present disclosure.

In another aspect, the invention includes treating the wound with an antimicrobial powder according to the present disclosure.

In aspects, the method of forming an antimicrobial film, non-woven, woven, gel, paste, or mesh according to the present disclosure may include spraying, blending, coating, immersion into an impregnation bath, and/or combinations thereof of the said antimicrobial composition. The method may further include pre-mixing and/or blending the antimicrobial composition according to the present disclosure with the components of the said film, non-woven, woven, gel, paste, or mesh prior to the formation of the said film, non-woven, woven, gel, paste, or mesh.

In another aspect, a method of forming an antimicrobial film, non-woven, woven, or mesh according to the present disclosure may include treating the said film, non-woven, woven, or mesh with an antimicrobial agent according to the present disclosure. The method of treating may include adding, blending, compounding, and/or mixing the antimicrobial agent(s) and/or antimicrobial compositions according to the present disclosure with the components of the said film, non-woven, woven, or mesh prior to the formation of the said film, non-woven, woven, or mesh.

In aspects, a method of preparing an antimicrobial film, gel, or paste on a surface may include the steps of: a. preparing a mixture of an antimicrobial composition in accordance with the present disclosure; b. optionally, adding at least one solvent and/or fluid to the mixture to form an intermediate mixture; c. applying the mixture and/or the intermediate mixture to the surface, and; d. curing, gelling, cooling, heating, radiating and/or drying the mixture obtained from step c, thereby obtaining an antimicrobial film and/or layer on the surface, wherein the surface may be a medical device and/or a mammalian tissue.

In aspects, the method of preparing the antimicrobial film, gel, or paste on a surface according to the present disclosure, wherein the surface may be the surface of a medical device may be a catheter, a fixation tape, a wound cover dressing, an absorbent wound dressing, an adhesive, a needle, a tube, a surgical instrument, a tape, an implant, a mask, a scaffold, an ostomy appliance, a collection bag, and combinations thereof.

In yet another aspect of the present invention, the antimicrobial composition is an antimicrobial foam or sponge that includes at least one antimicrobial agent in accordance with the present disclosure, wherein the antimicrobial agent may be covalently, ionically, and/or physically bound to the foam or sponge. The foam or sponge may include hydrophilic and/or hydrophobic foam or sponge. Further the foam or sponge may be open-celled, closed-celled, and/or combinations thereof.

The foam or sponge can be based on polymers selected from, but not limited to: silicone and/or its copolymers, polyurethane and/or its copolymers, collagen and/or its derivatives and copolymers, gelatin and/or its derivatives and copolymers, cellulose and/or its derivatives and copolymers, polyacrylic acid and/or its copolymers and salts, chitosan and/or its derivatives, polyvinyl alcohol and/or its copolymers, and combinations thereof. The foam or sponge may include additional components such as wound healing agents, surfactants, growth factors, antibiotics, hydrophilic additives, pH-buffering agents, and combinations thereof.

In certain embodiments, the invention is directed to an antimicrobial polyurethane foam comprising the reaction product of a polyisocyanate component and a polyol component, and further comprising an antimicrobial agent, wherein the antimicrobial agent comprises N^{α}-lauroyl-arginine ester or a salt thereof, for example, N^{α}-lauroyl-arginine ethyl ester or a salt thereof. Polyurethane foams can be formed by reacting a di- or polyisocyane with a polyol. Preparation of polyurethane foams, and foams with antibacterial agents are described in EP1964580B1 titled Silver-containing foam structure, U.S. Pat. No. 9,364,577 B2 and U.S. Pat. No. 8,946,315, the contents of each of which are incorporated by reference herein. The antimicrobial agent can, for example, be pre-dissolved in a suitable solvent or added as a powder to one of the reactant pre-mixture. Due to the presence of the free-amino group in the antimicrobial agent, the agent can be added to the surfactant or polyol solution phase or the catalyst phase, if it is a separate solution. Another method for the preparation of antimicrobial foams with antimicrobial agents of the present disclosure can include mixing the polyisocyanate component, surfactant/polyol component and antimicrobial solution as a separate component together prior to casting the mixture on a liner or carrier and allowing the composition to foam. These examples are to be considered non-limiting, and additional methods of incorporating the antimicrobial agents can be envisioned by one skilled in the art. In certain embodiments, the reaction product is present in an amount of about 95 to about 99.5% by weight of the composition and the N^{α}-lauroyl-arginine ester or a salt thereof is present in an amount from about 0.1 to aboutl0%, or 0.1 to about 5%, or about 0.2 to about 5% by weight of the composition. In yet additional aspects, the N^{α}-lauroyl-arginine ester or a salt thereof is present in an amount from about 0.1 to about 4% by weight of the composition. In yet further aspects, the N^{α}-lauroyl-arginine ester or a salt thereof is present in an amount from about 0.1 to about 3% by weight of the composition. In additional aspects, the N^{α}-lauroyl-arginine ethyl ester or a salt thereof is present in an amount of about 0.5% by weight of the composition. The foam can optionally further comprise a component selected from the group consisting of wound healing agents, surfactants, growth factors, antibiotics, hydrophilic additives, pH buffering agents, and combinations thereof. The invention also encompasses a wound dressing comprising a skin adhering region and an absorbent region, wherein the absorbent region comprises the foam described herein.

In another aspect, the invention includes a process for producing an antimicrobial foam or sponge, wherein said process may include treating the foam or sponge with a powder, solution, hotmelt, dispersion, emulsion, and/or suspension of the antimicrobial agent. As a non-limiting example, a hydrophilic polyurethane foam such as MEDISPONGE® SUPERSOFT™ (Essentra Porous Technologies), or SAQ Standard (from INOS Technologies), or ADMEDSOL foam (from Advanced Medical Solutions, B. V.) Product 1012 (from Polymer Health Technology) may be treated with a solution of the antimicrobial agent(s), such as Aminat G (LAE+glycerol) from Vedeqsa Inc., or CytoGuard LA 20 (from A&B Ingredients); or epsilon-polylysine solution such as 25% solution of E-polylysine (from Chisso Corporation). The foam may be soaked, immersed or impregnated with one or more antimicrobial agent and/or antimicrobial composition according to the present disclosure, followed by drying, curing, or heating the resulting foam to form the antimicrobial foam. Similar processes may be followed for polyvinylalcohol or silicone foam and/or sponge. In aspects, the antimicrobial agents and/or antimicrobial composition according to the present disclosure may be added, blended, and/or mixed with the components that may be used to form the foam or sponge. The foam or sponge can be surface treated or impregnated with the antimicrobial agents and/or antimicrobial composition according to the present disclosure.

In certain embodiments, the foam can be prepared by treating a foam with N^{α}-lauroyl-arginine ester or a salt thereof, wherein the foam comprises the reaction product of a polyisocyanate and a polyol component.

In certain aspects, the prepolymers of the polyurethane foam can be mixed with the antimicrobial agents and/or antimicrobial composition according to the present disclosure, and then the foam formed with the inclusion of said the antimicrobial agents and/or antimicrobial composition in the foam. For example, the process for producing the antimicrobial foam or sponge comprises producing the foam from a reaction mixture comprising a polyisocyanate component, a polyol component and N^{α}-lauroyl-arginine ethyl ester or a salt thereof. For example, the process for producing an antimicrobial polyurethane foam comprises the steps of reacting a polyisocyanate component and a polyol component in the presence of N^{α}-lauroyl-arginine ester or a salt thereof. The present invention also encompasses a composition for producing the antimicrobial polyurethane foam comprising: a polyisocyanate component; a polyol component; and N^{α}-lauroyl-arginine ester or a salt thereof.

In aspects, the process for producing the antimicrobial foam or sponge according to the present disclosure may include spraying, blending, coating, immersion into an impregnation bath, and/or combinations thereof of the antimicrobial agent according to the present disclosure.

In aspects, the process for producing the antimicrobial foam or sponge can comprise pre-mixing and/or blending the antimicrobial agent with the polymer prior to the formation of said foam or sponge.

In further aspects of the present disclosure, the antimicrobial compositions in accordance with the present disclosure can be prepared in the form of layers and/or surface having different thicknesses, morphologies, patterns, domains, functionalities, or the like, using any suitable processing techniques. Non-limiting examples of such processing techniques may include printing, extruding, calendering, molding, brushing, spraying, casting, coating, and/or application by hand. In aspects, the base layer or surface could the neat polymer, oligomer and/or an adhesive according to the present disclosure, followed by a layer or surface of the antimicrobial composition, which maybe further coated with a hydrophilic, hydrophobic, and/or amphiphilic layer. The coating may be a solution, an emulsion, suspension, dispersion, and combinations thereof or the like. In additional aspects, the invention includes a medical foam including at least one foamable and/or foamed composition, and at least one active agent selected from antimicrobial agent, growth factors, enzymes, polypeptides, proteins, lipids, polysaccharides, stem cells, antibiotics, stimulants, non-wound adhering agent and/or treatment, and the like. The polypeptides and proteins may include collagen, gelatin, elastin, pepsin, fibrin, and the like. The enzymes may include lipases, proteases, metallo-matrix proteases, collagenases, amylases, and the like. Further the foam may also include non-wound adhering agent and/or treatment including slip agents. In another aspect, the foam may include the foamed article and slip agent. The non-wound adhering treatment and/or slip agent may include glycerol monolaurate, and/or surfactants based on long carbon-chain (C6-C18) alkyl chains. Non-limiting example includes lauryl sulfate.

In another aspect, a medical substrate including at least one substrate selected from woven and non-woven fabric, mesh, absorbent fiber web, and combinations thereof; and at least one active agent selected from antimicrobial agent, growth factors, enzymes, polypeptides, proteins, lipids, polysaccharides, stem cells, antibiotics, stimulants, non-wound adhering surface treatment and/or agent, and the like. The polypeptides and proteins may include collagen, gelatin, elastin, pepsin, fibrin, and the like. The enzymes may include lipases, proteases, metallo-matrix proteases, collagenases, amylases, and the like. The non-wound adhering treatment and/or agent may include glycerol monolaurate, and/or surfactants based on long carbon-chain (C6-C18) alkyl chains. Non-limiting example includes lauryl sulfate.

In an additional aspect, a medical foam and/or sponge according to the present disclosure may include natural and/or synthetic polymers; further the natural and/or synthetic polymers may be selected from collagen, gelatin, chitosan, peptidoglycans, beta-glucans, polysaccharides, polypeptides, silicones, polyurethanes, polyvinyl alcohol, polyesters, polyamides, silicones and combinations thereof. The foam according to the present disclosure may further include plasticizing agents for the foam matrix rendering the structure soft and pliable. This may help conforming to the wound, skin substitution site, burn, Intravenous (IV) or catheter insertion sites. The plasticizing agents maybe added during the foaming process or after the foaming process. Non-limiting example of plasticizing agent may include glycerol, fatty acid esters, polyalkylene glycols, alkyl esters, and the like. In another aspect, the foam matrix may include the plasticizing or softening (lower the glass transition temperature or Tg) agent chemical bound to the matrix. Copolymers such as polyvinyl alcohol-ethylene oxide and/or polyvinylalcohol-vinyl acetate-vinylmethyl ether may be suitable examples.

In additional aspects, the invention includes a medical foam including at least one foamable and/or foamed composition, and at least one active agent selected from antimicrobial agent, growth factors, enzymes, polypeptides, proteins, lipids, polysaccharides, stem cells, antibiotics, stimulants, non-wound adhering agent and/or treatment, and the like. The polypeptides and proteins may include collagen, gelatin, elastin, pepsin, fibrin, and the like. The enzymes may include lipases, proteases, metallo-matrix proteases, collagenases, amylases, and the like. Further the foam may also include non-wound adhering agent and/or treatment including slip agents. In another aspect, the foam may include the foamed article and slip agent. The non-wound adhering treatment and/or slip agent may include glycerol monolaurate, and/or surfactants based on long carbon-chain (C6-C18) alkyl chains. Non-limiting example includes lauryl sulfate.

In another aspect, a medical substrate including at least one substrate selected from woven and non-woven fabric, mesh, absorbent fiber web, and combinations thereof; and at least one active agent selected from antimicrobial agent, growth factors, enzymes, polypeptides, proteins, lipids, polysaccharides, stem cells, antibiotics, stimulants, non-wound adhering surface treatment and/or agent, and the like. The polypeptides and proteins may include collagen, gelatin, elastin, pepsin, fibrin, and the like. The enzymes may include lipases, proteases, metallo-matrix proteases, collagenases, amylases, and the like. The non-wound adhering treatment and/or agent may include glycerol monolaurate, and/or surfactants based on long carbon-chain (C6-C18) alkyl chains. Non-limiting example includes lauryl sulfate.

In an additional aspect, a medical foam and/or sponge according to the present disclosure may include an active agent, wherein the active agent maybe dispersed within the cavities or cells or along the cell wall of the foam or sponge. The foam or sponge may be porous, reticulated, open and/or close cell.

In aspects, an antimicrobial composition according to the present disclosure includes at least one or more antimicrobial agent selected from: natural polypeptides, N-acylamino acid esters and/or their salts, esters of glycerol and saturated and/or unsaturated fatty acids (C₆-C₂₀), saturated and/or unsaturated alcohols with C₆-C₂₀ carbon atoms, and combinations thereof; wherein the antimicrobial agent is present in an amount 0.5-90.0 wt %, 5.0-80.0 wt %, or 10.0-70.0 wt %.

The antimicrobial composition according to the present disclosure may be present in the form selected from liquids, gels, creams, foams, lotions, paste, powder, aerosols, and combinations thereof.

The antimicrobial composition according to the present disclosure may further include at least chelating agent, present in an amount 0.01-10 wt %, 0.05-5.0 wt %, or 0.1-3.0 wt %.

The chelating agent according to the present disclosure may be selected from the group of ethylenediaminetetraacetic acid (EDTA), diethylenetriaminepentaacetic acid, 2 hydroxyethylethylene-diamine-triacetic acid, 1,6-diaminohexamethylenetetraacetic acid, 1,2-diamino-cyclohexanetetraacetic acid, O,O′-bis(2-aminoethyl)ethyleneglycoltetraacetic acid, 1,3-diaminopropanetetraacetic acid, N,N′-bi s(2-hydroxybenzyl) ethylenediamine-N,N′-diacetic acid, ethylenediamine-N,N′-diacelic acid, ethylenediamine-N,N′-dipropionic acid, triethylenetetraaminehexaacetic acid, ethylenediamine-N,N′-bis(methylenephosphonic acid), iminodiacetic acid, N,N-bis(2-hydroxyethyl)glycine, 1,3-diamino-2-hydroxypropanetetraacetic acid, 1,2-diaminopropanctetraacetic acid, ethylenediaminetetrakis(methylenephosphonic acid), N-(2-hydroxyethyl)iminodiacetic acid, biphosphonates, poly(maleic acid) and its copolymers, poly(maleic anhydride) copolymers, poly(citric acid), polycitrates, polyglutamic acid, polyaspartic acid, poly(succinimide), poly(allylamine) and its copolymers, poly(diallydimethyl ammonium chloride) (polyDADMAC), polyamidoamine (PAMAM) and its copolymers, polyvinylpyrolidone, polystyrenesulfonic acid and/or its salts, poly(styrenesulfonic acid-maleic acid) copolymer and/or its salts, polyacrylic acid and/or its salts, polyacrylic acid copolymers and/or their salts, sulfonated polystyrene and/or its copolymers, and/or their salts, polycitric acid and/or its copolymers, and/or their salts, poly(isobutylene-maleic anhydride) copolymer and/or its salts, polyethyeleneimine and/or its copolymers and/or salts, polyoxazoline and its copolymers and/or salts, hyaluronic acid and its derivatives, chitosan, and combinations thereof

The antimicrobial composition described herein may prevent the regrowth of biofilm organisms for at least 24 hours after treatment with said antimicrobial composition.

The antimicrobial composition according the present disclosure may kill at least 90% of microbes after exposure to said antimicrobial composition for 24 hours.

The adhesive or antimicrobial composition according to the present disclosure may further include surfactants, hydrophilic additives, pH-buffering agents, solvents, thickening agents, and combinations thereof. The thickening agents may be used to alter the viscosity of the composition when presented as a liquid.

The thickening agent may be non-ionic, anionic, cationic, amphoteric or combinations thereof present in an amount of 0.1-50.0 wt %, 0.5-30.0 wt %, or 1.0-20.0 wt %, and may be selected from polyvinylpyrolidone, polystyrenesulfonic acid and/or its salts, polystyrenesulfonic acid-alt-maleic acid and/or its salts, polyalkyleneoxide and/or its copolymers, polyacrylic acid and its copolymers and/or its salts, gums, chitosan, polysaccharides, polypeptides, hydrocolloids, nanoclays, polyacrylamide and its copolymers and/or its salts, and combinations thereof.

The antimicrobial composition according to the present disclosure may be used to prepare wound cleansers, used in combination with debriding, use to treat or prevent infection and/or biofilm regrowth or formation.

In an additional aspect of the present disclosure, the antimicrobial composition is an antimicrobial solution or cleanser to clean and/or disinfect the affected tissue in a mammalian body may include the antimicrobial agents according to the present disclosure. In another aspect, the solution or cleanser may be incorporated on or in a non-woven, cloth, fabric, and the like, which may be used to clean and/or disinfect the affected tissue. Such antimicrobial wipes are commonly used in patient care. Such cleaning or disinfecting options are typically used between dressing changes, and to address potential issues of infection on a wound or skin. The wounds may be cuts, mechanical wounds, surgical wounds, burn wounds, ulcerous, fistula, and the like. Further, the antimicrobial solution or cleanser may be used to debride and/or irrigate the wound or affected tissue. In another aspect, the cleaning liquid or solution may not include a surfactant as some patients may be sensitive to such chemicals. The antimicrobial cleansing compositions may include at least one antimicrobial agent according to the present disclosure and saline and/or water. Optionally, the composition may include moisturizing agents, humectants, vitamins, enzymes, enzyme cofactors, wound healing agents such as honey, and the like.

In certain aspects, the cleanser is an aqueous antimicrobial composition comprising:
a. N^{α}-lauroyl-arginine ester or a salt thereof in an amount between about 0.01 to about 1% by weight of the composition;
b. glycerol in an amount between about 0.1 to about 10%.

In certain embodiments, the N^{α}-lauroyl-arginine ester or salt thereof is N^{α}-lauroyl-arginine ethyl ester or a salt thereof. The N^{α}-lauroyl-arginine ethyl ester of salt thereof can, for example, be present in an amount between about 0.02 to about 0.7% by weight of the composition. In certain embodiments, the composition further comprises a coconut oil-based surfactant, for example, in an amount between about 0.2 to about 2% by weight. In certain aspects, the composition comprises a chelating agent such as those described herein and including, for example, EDTA or a salt thereof such as a sodium salt of EDTA. In certain additional aspects, the composition further comprises sorbitol and Polysorbate 20. In yet additional aspects the composition does not comprise an antimicrobial agent selected from the group consisting of silver and salts thereof (for example, silver sulfadiazine), chlorohexidine gluconate (CHG), polyhexamethylenebiguanide (PHMB), iodine, hyperchlorous acid and/or octenidine dihydrochloride. An exemplary aqueous composition comprises the components in the amounts shown in the Table below:

TABLE A

| Component | Amount (% by weight of composition) |
|---|---|
| Disodium EDTA | 0.05 |
| Sorbitol | 1.5 |
| Disodium cocamphodiacetate | 0.5 |
| Polysorbate 20 | 0.1 |
| N^{α}-lauroyl-arginine ester | About 0.02 to about 0.7 |
| Glycerol | About 0.1 to about 3.5 |

As discussed above, the cleanser described herein can be incorporated into an antimicrobial wipe; for example, the composition can be incorporated on or in a non-woven, cloth, fabric, and the like, which may be used to clean and/or disinfect an affected tissue.

The adhesive and antimicrobial compositions (specifically including, for example, the antimicrobial adhesive compositions, the gels, and the cleansers) described herein can be used to treat a wound in a patient in need thereof. In addition, the invention encompasses a method of treating a wound in a subject in need thereof, wherein the wound is at risk for infection, comprising treating the wound with a composition comprising an antimicrobial amount of N^{α}-lauroyl-arginine ester or a salt thereof, for example, N^{α}-lauroyl-arginine ethyl ester or a salt thereof. The wound can, for example, be treated after a wound dressing is removed, before a wound dressing is administered and/or between changes of wound dressings. In some embodiments, the invention encompasses a method of treating a wound in a subject in need thereof, wherein the wound is at risk for infection, for example, bacterial and/or fungal infection, comprising treating the wound with a composition comprising an antimicrobial amount of N^{α}-lauroyl-arginine ethyl ester or a salt thereof. In certain aspects, the composition further comprises a humectant. In certain aspects, the humectant is glycerol. In yet additional aspects the composition further comprises a coconut oil-based surfactant. Such coconut oil-based surfactants include, for example, disodium cocamphodiacetate, coco-betaine, an amino acid derivative of coconut oil, or a phospholipid derivative of coconut oil. The coconut oil-based surfactant, such as disodium cocamphodiacetate, can be present in the composition in an amount of about 0.2 to about 2% by weight of the composition.

In certain aspects, the antimicrobial compositions and the methods described herein comprise the use of N^{α}-lauroyl-arginine ester or a salt thereof and/or N^{α}-lauroyl-arginine ethyl ester in an effective amount, for example, in an antimicrobial amount. The antimicrobial amount of the N^{α}-lauroyl-arginine ester is an amount effective in providing an antimicrobial effect in vivo or in vitro. Methods of determining an antimicrobial effect are described in detail and in the Examples. In certain aspects, the antimicrobial effect can be tested or measured as described herein, for example, by providing a zone of inhibition and/or reducing the number of colony forming units (CFUs). For example, an antimicrobial amount of an agent is the amount or dose of the agent that reduces the number of colony forming units (CFUs) as compared to that in the absence of the treatment. In yet additional aspects, the antimicrobial amount is the amount effective to reduce the number of CFUs by at least about one order of magnitude after about 24 hours of exposure; in yet further aspects, the antimicrobial amount is the amount effective to reduce the number of CFUs of Staphylococcus aureus, Pseudomonas aeruginosa, E. coli, Aspergillus brasiliensis, Methicillin-resistant Staphylococcus aureus (MRSA), C. albicans, and/or aspergillus niger by at least one order of magnitude after about 24 hours of exposure. In yet further embodiments, the antimicrobial amount of N^{α}-lauroyl-arginine ethyl ester is between about 0.01 to about 5% by weight; between about 0.01 to about 3% by weight of the composition; between about 0.01 to about 2% by weight of the composition; or between about 0.01 to about 1% by weight of the composition. In certain embodiments, the methods described herein reduce the number of colony forming units (CFUs) of microbes by at least one log order after about 24 hours of treatment. In further aspects, the method reduces the number of colony forming units (CFUs) of microbes by at least about 60% after an exposure time of about 5 minutes. In yet additional aspects, the microbe is selected from the group consisting of Staphylococcus aureus, Pseudomonas aeruginosa, E. coli, Aspergillus brasiliensis, Methicillin-resistant Staphylococcus aureus (MRSA), C. albicans, and/or aspergillus niger, or any combination thereof. In certain additional aspects, the number of CFUs of aspergillus niger is reduced by at least one log order after about 24 hours of treatment.

In another aspect, a wound and/or skin care dressing including a substrate and at least one hydrophilic silicone adhesive according to the present disclosure, wherein the hydrophilic silicone adhesive further includes at least one humectant and at least one silicone adhesive. The silicone adhesive may be a crosslinked, branched, linear polymers, pressure sensitive adhesive, gel adhesive, and/or combinations thereof.

In yet an additional aspect, the antimicrobial composition is an antimicrobial tissue substitute or scaffold that comprises at least one tissue substitute material and at least one antimicrobial agent. In an additional aspect, the antimicrobial tissue substitute or scaffold is a skin substitute or scaffold and can include at least one skin substitute material and at least one antimicrobial agent. In certain aspects, the antimicrobial tissue substitute or scaffold is non-cytotoxic. In certain aspects, the antimicrobial tissue substitute or scaffold reduces the number of colony forming units (CFUs) of microbes by at least one log order after about 24 hours of treatment. The antimicrobial tissue substitute or scaffold, can for example, be prepared by a method comprising treating the tissue substitute or scaffold with an antimicrobial agent prior to use on a wound, wherein the antimicrobial agent is selected from the group consisting of ε-polylysine and N^{α}-lauroyl-arginine ester or a salt thereof, or a combination thereof. In certain aspects, N^{α}-lauroyl-arginine ester or a salt thereof is the N^{α}-lauroyl-arginine ethyl ester or a salt thereof. In an additional aspect, the antimicrobial tissue substitute or scaffold is prepared by a method comprising treating the tissue substitute or scaffold with an antimicrobial agent during the manufacture of the tissue substitute or scaffold, wherein the antimicrobial agent is selected from the group consisting of ε-polylysine and N^{α}-lauroyl-arginine ethyl ester or a salt thereof, or a combination thereof.

In such applications, it is important to reduce the bioburden on a compromised tissue. This may be achieved by incorporating antimicrobial agents in the tissue substitute or scaffold. Optionally, the tissue substitute or scaffold can be treated prior to use with the antimicrobial composition according to the present disclosure. In some instances, the tissue substitute or scaffold may also include a synthetic polymer film or layer or membrane to protect the skin substitute from external environment and also to provide a visual for the medical care giver to check the underlying skin growth. An example of such a commercial product is INTEGRA® Dermal Regeneration Template, which is a two-layer skin regeneration system, where the outer layer is made of a thin silicone film, and the inner layer is constructed of a complex matrix of cross-linked fibers. In such instances, the antimicrobial composition according to the present disclosure may be included in both layers of the skin substitute system. In certain embodiments, the antimicrobial agent is N^{α}-lauroyl-arginine ester or a salt thereof, for example, N^{α}-lauroyl-arginine ethyl ester or a salt thereof. In yet additional aspects, the antimicrobial is polylysine, for example, c-polylysine. In yet further aspects, the antimicrobial is a composition comprising N^{α}-lauroyl-arginine ethyl ester and ε-polylysine. The tissue substitute material can be biologic, natural and/or synthetic material. Several classes of tissue substitutes can be used, such as: Temporary impervious dressing materials including naturally occurring or biological dressing substitute, non-limiting examples include amniotic membrane, potato peel; or synthetic dressing substitute, for example synthetic polymer sheet including polyurethanes, silicones, polyvinylalcohol, and the like; bi-layered tissue engineered materials, non-limiting example includes TRANSCYTE®; Single layer durable skin substitutes such as Epidermal substitutes, for example cultured epithelial autograft (CEA), collagen sheets wherein the collagen may be ovine, bovine, porcine, and/or human origin; Composite skin substitutes including allograft, xenograft; Tissue-engineered skin selected from amniotic tissue, placental tissue, collagen and/or its derivatives, and combinations thereof. The antimicrobial tissue substitute can be delivered, for example, as powder, gel, liquid, dressing, film, mesh, and the like. In yet additional embodiments, the tissue substitute or scaffold comprises collagen, gelatin, and/or amniotic membrane, and is treated with an antimicrobial agent, for example N^{α}-lauroyl-arginine ethyl ester or a salt thereof, ε-polylysine, or a combination thereof. In certain aspects, the N^{α}-lauroyl-arginine ethyl ester is present in amount between about 0.01 to about 5% by weight.

The skin substitute or scaffold described herein can be used, for example, in the treatment of deep dermal and full thickness wounds. Such wounds include, for example, burns.

In another aspect, an antimicrobial skin substitute according to the present disclosure may include a natural polypeptide such as polylysine and/or nisin, and combinations thereof. Such natural polypeptides have a dual function of promoting cell growth and reducing bioburden or being antimicrobial or preventing microbial growth. In another aspect, an antimicrobial skin substitute according to the present disclosure may include an antimicrobial agent bound chemically or physically to skin substitute materials disclosed in the present disclosure.

In another aspect, the invention includes an antimicrobial medical device including at least one antimicrobial agent according to the present disclosure including natamycin or pimaricin as fungicide. The medical device may be a wound care or skin care device, catheters, stents, cardiac or orthopedic or ocular implants, and the like.

As described above, the antimicrobial skin or tissue substitute and/or scaffold according to the present disclosure is non-cytotoxic. Non-cytotoxicity can be determined, for example, as per ISO 10993 tests. The cytotoxicity (or lack thereof) of the antimicrobial tissue substitute or scaffold can be an important characteristic of the product. The major function of these substitutes and scaffolds is the promotion of cell growth in order to heal the affected area such as wound or lost tissue. Commonly used antimicrobial agents such as PHMB and silver can be cytotoxic depending on their concentrations to achieve antimicrobial effect. One potential advantage of the antimicrobial agents described herein (such as N^{α}-lauroyl-arginine ethyl ester or a salt thereof and/or ε-polylysine) is that they can be used as levels to provide antimicrobial effect while being non-cytotoxic to cells, thereby allowing cell growth and proliferation.

As described above, the antimicrobial compositions of the present disclosure can be tested for antimicrobial effect using numerous techniques known to those having ordinary skill in the art. Non-limiting examples of such tests include zone of inhibition (ZOI or corrected ZOI (CZOI)) test, kill rate (log-reduction) test over time per ASTM E 2315-03. 2008 Standard Guide for Assessment of Antimicrobial Activity using a Time-Kill Procedure, and Clinical and Laboratory Standards Institute, Vol 19 No. 18, 1999. M26-A. Methods for Determining Bactericidal Activity of Antimicrobial Agents: Approved Guideline; anti-biofilm capabilities using Calgary Biofilm Method (ASTM E2799), ISO 22196:2007, ISO 22196:2011, combinations thereof, or the like. Other tests may include minimum inhibitory concentration (MIC) and minimum bacteriocidal concentration (MBC).

The ZOI or CZOI method involves placing a piece (for example, a 1 inch by 1 inch piece) of the antimicrobial composition or article (d=20-25 mm) on an agar surface (Muller Hinton agar (MH agar)) in 25 ml/9 cm plates that produce an agar depth of 4 mm, which has been seeded with the test microorganism (Staphylococcus aureus, Pseudomonas aeruginosa , and Candida albicans) for a given length of time, for example 24-hours. Diffusion of antimicrobial agent into the agar results in inhibition of growth, which appears as a clear or hazy zone on the agar. The diameter of the whole inhibition zone may be termed as the zone of inhibition, and the corrected zone of inhibition may be determined as the diameter of the whole inhibition zone minus the size of the antimicrobial composition or article. In aspects, other medium for growth of the microbial species may be used, such as, for bacteria, a cation adjusted Muller Hinton Agar (CAMHA), and for yeast sabouraud dextrose Agar (SDA).

Other tests for antifungal and antibacterial properties may also be included. The antimicrobial compositions according to the present disclosure are expected to reduce the colony forming units (CFU) by at least one order of magnitude during 24 hours of exposure. The antimicrobial compositions according to the present disclosure are expected to reduce biofilm regrowth versus a non-treated control.

For biofilms testing, the biofilms may be established and assayed by techniques known to those skilled in the art. Biofilm testing is described in U.S. Pat. No. 8,829,053 B2, the contents of which are incorporated by reference herein. For example, for each organism, 96-peg MBEC™ pegs may be placed in a 96 well plate with 100 pl of 0.1 OD 600 log phase bacterial culture per well. The biofilms are then allowed to grow on these pegs for 36 to 48 hours. Then the excess bacteria may be rinsed off in a 96 well plate with phosphate buffer saline (PBS) for about 10 minutes. The cells may then be treated with the antimicrobial compositions and positive and negative controls in a 96 well plate at for about 8 minutes. The plates may then be rinsed as above in a fresh plate. The pegs may then be transferred to a neutralization plate containing Dey-Engley broth and lightly sonicated for about 10-15 mins to release the planktonic organisms associated with the pegs. After sonication, the peg plate may be moved to a regrowth plate with Tryptic Soy Broth (TSB) and incubated for about 24 hours. The assay may be completed by reading the absorbance at 600 nm in a microplate reader such as Molecular Devices M2. The above biofilm test may be modified or other suitable tests may be used.

The invention is illustrated by the following examples which are not meant to be limiting in any way.

### EXEMPLIFCATION

The examples described herein can be modified without departing from the scope of the present disclosure.

The following materials were used to prepare the antimicrobial composition of the present disclosure: Silpuran 2130 A/B from Wacker Chemie AG, NaCMC (Aqualon 7HF Pharma from Ashland Inc.), Aminat G from Vedeqsa Inc., Epsiliseen-H (epsilon-polylysine) from Siveele B.V., E-polylysine (50:50 blend of dextrin and epsilon-polylysine from DKSH North America, Inc. (Chisso Corporation), Lauricidin from Med-Chem Labs Inc., Lauryl arginate ethyl ester hydrochloride (LAE or LAE.HCl) from A&B Ingredients; Glycerol ethoxylate, Glycerol, Gelatin, Xanthan gum from Sigma-Aldrich; Rice protein from Whole Earth, Princeton, N.J.; Polyurethane film EU28 from Delstar, and polycarbonate liner from Wiman Corporation. Monolaurin from Colonial Chemical, Inc. Cytoguard LA20 from A&B Ingredients. Hydroxyethyl cellulose (Natrosol 250HH Pharma) and Klucel JF from Ashland Chemical. PEG-8 from Croda International Plc. Hydrolite 5 from Symrise Group, Crodateric CDA 40 from Croda International Plc. ColaLipid C from Colonial Chemical Inc. Acrylic adhesive DURO-TAK® 129A™ and DURO-TAK® 3053™ from Henkel Corporation.

### Example 1

### Antimicrobial Silicone Adhesive Compositions and Zone of Inhibition (ZOI) Testing

General procedure: The silicone adhesive components, Parts A and B were weighed out in a plastic jar, and then the rest of the ingredients were added. The mixture was thoroughly mixed with a stainless steel spatula, and then coated onto a polyurethane film to a specified thickness (8-10 mils or about 200-250 grams per square meter) using film-casting knife from Byk Instruments. The coated adhesive was then cured at 130° C. for 4 minutes. The cured adhesive on film was removed from the oven and the adhesive surface protected with a polycarbonate liner. The tack or adhesiveness of the surface for the antimicrobial adhesive can be evaluated by dry thumb test, wherein the clean and dry thumb (cleaned with isopropanol solution and dried) may be placed on the surface of the adhesive with gentle pressure and the thumb removed within a few seconds (less than one minute). The ease of thumb removal indicates the tackiness or adhesiveness of the adhesive. Table 1 lists the antimicrobial adhesive compositions, 1-6, along with a Control (neat silicone gel), along with the resulting tack test results as ‘cured adhesive properties’.

ZOI testing of antimicrobial adhesive compositions: The antimicrobial adhesive compositions as listed in Table 1, which were coated on polyurethane film, and then surface protected by polycarbonate liner, were cut into 1 inch×1 inch strips. The strips were then evaluated for antibacterial and antifungal activity by Agar diffusion susceptibility method, after removing the protecting polycarbonate liner, and then exposing the adhesive surface to the cultured medium with bacteria or yeast or fungus. The following guidelines were used:

Bacteria (Staphylococcus aureus; strain—ATCC6538; Pseudomonas aeruginosa; strain—ATCC15442); Fungus (Candida albicans; strain—ATCC10231). The following methods were used: Bacterial: Methods for dilution antimicrobial susceptibility tests for bacteria that grow aerobically; Fungal: Reference method for broth dilution antifungal susceptibility testing of yeasts; Method: Agar diffusion method. The following medium were used: Bacteria: Cation Adjusted Muller Hinton Agar (CAMHA); Yeast (or Fungus): Sabouraud Dextrose Agar (SDA). The test was repeated in triplicates per composition, and the end point was Zone of Inhibition (mm) as measured by clear and/or hazy zone after 24 hours of exposure for bacteria and 48 hours of exposure for Candida albicans. The thickness of the medium was 4 mm. Table 2 shows the results of the ZOI test for the different compositions 1 through 6 and Control as an average of 3 readings.

TABLE 1
Antimicrobial adhesive compositions

| | Weight % composition | | | | | | |
|---|---|---|---|---|---|---|---|
| Components | Control | 1 | 2 | 3 | 4 | 5 | 6 |
| Silpuran 2130 Part A | 50 | 41 | 27.3 | 18.2 | 31.8 | 31.8 | 29.5 |
| Silpuran 2130 Part B | 50 | 49 | 32.7 | 21.8 | 38.2 | 38.2 | 35.5 |
| Aqualon 7HF Pharma | — | — | — | — | 20 | — | 5 |
| Aminat G | — | — | — | — | 10 | 10 | — |
| E-polylysine | — | 20 | 40 | — | — | 20 | — |
| Epsiliseen-H | — | — | — | 60 | — | — | — |
| Lauricidin | — | — | — | — | — | — | 10 |
| Glycerol ethoxylate | — | — | — | — | — | — | 20 |
| Cured adhesive properties | High tack | High tack | Medium tack | Very low tack | High tack | High tack | High tack |

TABLE 2
Zone of inhibition results for antimicrobial
adhesive compositions of Table 1

| | Zone of Inhibition (mm, includes | | |
|---|---|---|---|
| | sample size of 25 mm × 25 mm) | | |
| | S. aureus | P. aeruginosa | C. albicans |
| Composition | (ATCC 6538) | (ATCC15442) | (ATCC10231) |
| Control | NZ | NZ | NZ |
| 1 | CZ (25 mm) | CZ (25 mm) | NZ |
| 2 | CZ (25 mm) | CZ (26 mm) | NZ |
| 3 | HZ (28 mm) | HZ (28 mm) | CZ (28-37 mm) |
| 4 | CZ (27 mm) | HZ (37 mm) | spotty growth* |
| 5 | CZ (28 mm) | CZ (30 mm) | spotty growth* |
| | | HZ (39 mm) | |
| 6 | HZ (27 mm) | HZ (36 mm) | spotty growth* |

NZ = No zone of inhibition;
CZ = Clear zone of inhibition;
HZ = Hazy zone of inhibition
*spotty growth = clear zone of inhibition across plate with tiny spots of growth

As Table 2 shows, the neat silicone gel did not have any antimicrobial effect, as expected. The antimicrobial adhesive compositions according to the present disclosure, Compositions 1 and 2 seem to be effective against S. aureus and P. aeruginosa, and not against C. albicans. This could be due to the dilution effect of dextrin, which may have reduced the effective concentration of polylysine needed for inhibition. Composition 3 showed inhibitory effect for both bacterial species and the yeast, C. albicans. Since this is 100% epsilon-polylysine (not mixed with dextrin), it has high antimicrobial effect. Due to the high level of antimicrobial agent, the adhesive property was poor as exhibited by the very low tack to touch.

Composition 4 with N^{α}-lauroyl-arginine ester hydrochloride (LAE) showed inhibitory effect for all three species. Due to the basic group present in this compound, the addition-cure of the silicone gel seemed to be impacted, resulting in a cohesively weak adhesive gel that leaves residue on finger when touched. Surprisingly, this cure issue was improved by addition of E-polylysine as shown for Composition 5. In addition, the zone of inhibition seems to have synergistically impacted the inhibitory effect on both bacterial species while maintaining the same effect against yeast. Composition 6 with Lauricidin (lauroyl ester of glycerol or monolaurin) showed inhibitory effect against all three species. It should be noted that the resulting gel was very tacky, and also seemed to have a waxy layer on surface possibly due to the lauroyl group. This may be advantageous to release the composition from a surface on which the composition may be cured.

The antimicrobial silicone adhesive compositions including lauryl arginate ethyl ester hydrochloride having the components described in the Table 3 were prepared as described above. The adhesiveness of the formulations was observed and measured using the dry thumb tack test and the stainless steel peel test (ASTM D3330 Method A). After peel the adhesive tape from stainless steel panels, the residue level on the plates were assessed qualitatively. The results of these tests are shown below in Table 3.

This example describes two specific silicone gel adhesive formulations, and also describes the observed adhesive properties. The first example (Composition A) includes Silicone gel adhesive (Blend of Part A+Part B) at 85% by weight, Aminat-G at 10% by weight (Glycerol: 8%; LAE.HCl-2%) and Hydroxyethyl cellulose at 5% by weight. The second example (Composition B) includes Silicone gel adhesive (Blend of Part A+Part B) at 95% by weight and Mirenat-NSM at 5% by weight (Maltodextrin: ˜80%; LAE.HCl ˜20%).

TABLE 3
Antimicrobial silicone adhesive compositions and their adhesive properties

| Ingredients | Control | 1 | 2 | 3 | 4 | 5 |
|---|---|---|---|---|---|---|
| Silpuran 2130 Part A | 49 | 44.55 | 42.3 | 42.3 | 42.3 | 42.3 |
| Silpuran 2130 Part B | 49 | 53.45 | 50.7 | 50.7 | 50.7 | 50.7 |
| Aminat G | | | | | | |
| LAE HCl (neat) | 2 | 2 | 2 | 2 | 2 | 2 |
| Mirenat-NSM (80/20 blend of | | | | | | |
| maltodextrin/LAE•HCl) | | | | | | |
| NaCMC | | | | | | |
| Glycerol | | | | | | |
| HEC | | | | | 5 | |
| Rice protein | | | 5 | | | |
| Xanthan gum | | | | | | 5 |
| Gelatin | | | | | | |
| Cured adhesive properties | Poor cure - | Cured but | Cured well; | Cured well; | Cured but | Cured well; |
| (dry thumb tack test - placing | cohesively | some residue | high tack | med tack | poor cohesive | slight residue |
| clean and dry thumb on | weak | on finger tack | and | and | strength; very | in some |
| adhesive surface with | | test; very | adhesion | adhesion | leggy; high | areas; med |
| pressure and withdrawing | | leggy | | | residue on | tack and |
| thumb within few seconds; | | | | | finger tack | adhesion |
| evaluate residue on thumb) | | | | | test | |
| SS peel data (180-deg); | Not tested | 80 g/in; high | 15 g/in; No | 25 g/in; No | 70 g/in; high | 35 g/in; no |
| residue level; ASTM | | residue; | residue | residue | residue; | residue |
| D3330/D3330M-04, method A | | >50% | | | >50% transfer | |
| | | transfer to | | | to plate | |
| | | plate | | | | |

| Ingredients | 6 | 7 | 8 | 9 |
|---|---|---|---|---|
| Silpuran 2130 Part A | 42.3 | 38.6 | 41 | 42.3 |
| Silpuran 2130 Part B | 50.7 | 46.4 | 49 | 50.7 |
| Aminat G | | 10 | | |
| LAE HCl (neat) | 2 | | | 2 |
| Mirenat-NSM (80/20 blend of | | | 5 | |
| maltodextrin/LAE•HCl) | | | | |
| NaCMC | | | | 5 |
| Glycerol | | | 5 | |
| HEC | | | | |
| Rice protein | | | | |
| Xanthan gum | | 5 | | |
| Gelatin | 5 | | | |
| Cured adhesive properties | Cured well; | Cured well; | Cured well; | Cured well |
| (dry thumb tack test - placing | some areas | very small | high adhesion | but very |
| clean and dry thumb on | are leggy and | areas are | and tack; | tacky and |
| adhesive surface with | leave residue | leggy and | | leggy; |
| pressure and withdrawing | probably due | leave residue; | | release liner |
| thumb within few seconds; | to residual | med tack and | | adhesion |
| evaluate residue on thumb) | IPA solvent | adhesion | | tighert and |
| | | | | leaves |
| | | | | residue |
| SS peel data (180-deg); | 25 g/in; | 20 g/in; slight | Not tested | Not tested |
| residue level; ASTM | leaves some | residue on | | |
| D3330/D3330M-04, method A | residue; | plate; <10% | | |
| | <25% | transfer to | | |
| | transfer to | plate | | |
| | plate | | | |

### Example 2

### Antimicrobial Acrylic Adhesive Compositions

Antimicrobial acrylic adhesives were prepared by mixing acrylic adhesive solutions with the antimicrobial agents (see Table 4 below). As a control agent, Povidone-Iodine (from Ashland Specialty Chemicals) was also used. The mixtures were coated to 1-2 mil (or 25-50 microns) wet thickness using Meyer rod #20 on siliconized release paper. The coatings were first dried at room temperature for 10 minutes followed by 10 minutes at 200° F. The dried antimicrobial acrylic adhesive samples were tested for antimicrobial efficacy using ZOI test described in Example 1. Samples were run in triplicates for each strain and each composition. The results are shown in Table 4 below.

TABLE 4
Antimicrobial acrylic adhesives of the present disclosure

| Composition # | Acrylic adhesive (g) | Antimicrobial Agent (g) |
|---|---|---|
| 1 | DURO-TAK ® | Aminat-G - 3.8 g (0.76 g) |
| | 3053 ™ - 30 g | |
| 2 | DURO-TAK ® | Aminat-G - 3.8 g |
| | 129A ™ - 30 g | |
| 3 | DURO-TAK ® | Polylysine - 3 g |
| | 129A ™ - 75 g | |
| 4 | DURO-TAK ® | EtOH/GML (10% soln) - 1 g |
| | 129A ™ - 30 g | |
| 5 | DURO-TAK ® | Povidone-Iodine - 1 g |
| | 129A ™ - 30 g | |

TABLE 5
Zone of inhibition results for antimicrobial
acrylic adhesive compositions of Table 3

| | Zone of Inhibition (mm, includes | | |
|---|---|---|---|
| | sample size of 25 mm × 25 mm) | | |
| | S. aureus | P. aeruginosa | C. albicans |
| Composition | (ATCC 6538) | (ATCC15442) | (ATCC10231) |
| 1 | CZ (25 mm) | CZ (25 mm) | CZ (25 mm) |
| 2 | CZ (25 mm) | CZ (25 mm) | CZ (25 mm) |
| 3 | CZ (26 mm) | CZ (25 mm) | HZ (25 mm) |
| 4 | CZ (25 mm) | CZ (25 mm) | HZ (25 mm) |
| 5 | HZ (25 mm) | HZ (25 mm) | HZ (25 mm) |

CZ = Clear zone of inhibition;
HZ = Hazy zone of inhibition

The above antimicrobial adhesive compositions are expected to prevent biofilm growth after exposure to such compositions.

Further DURO-TAK® 129A™ 5 grams (50% solution) was mixed with Aminat-G (20% LAE in Glycerol) as follows:

TABLE 6
Additional Antimicrobial acrylic adhesive formulations

| Ingredients | Control | A | | B | | C | |
|---|---|---|---|---|---|---|---|
| DURO-TAK ® | 100% | 20.0 | gm | 20.0 | gm | 20.0 | gm |
| 129A ™ (50% | | | | | | | |
| solution) | | | | | | | |
| Aminat-G | — | 0.5 | gm | 1.0 | gm | 2.5 | gm |
| (20% LAE in | | | | | | | |
| glycerol) | | | | | | | |
| Stainless Steel | 1900 g/in | 1606 | g/in | 1667 | g/in | 1299 | g/in |
| Adhesion | | | | | | | |
| per ASTM | | | | | | | |
| D3330/D3330M- | | | | | | | |
| 04, method A | | | | | | | |

| | | | | |
|---|---|---|---|---|
| Log reduction of | No log | Expect | Expect | Expect |
| S. aureus, | reduction | at least | at least | at least |
| P. aeruginosa | expected | 1-log | 1-log | 1-log |
| per ASTM E | | reduction | reduction | reduction |
| 2315-03. 2008 | | | | |

### Example 3

### Antimicrobial Foam Compositions

Hydrophilic polyurethane foam from Freudenberg Performance Materials was treated with AMINAT-G® or Cytoguard LA20 at 20% wet weight of the total wet foam. The foam samples were dried for several days at room temperature. Also, the foam was sprayed with 20% solution of Epsiliseen-H (from Siveele B.V.). The weight of added solution was 38% of the total foam weight. This was dried for 48 hours at room temperature before testing for antimicrobial efficacy by ZOI test described above. The ZOI results are shown in Table 7 below:

TABLE 7
Zone of Inhibition studies on Freudenberg
hydrophilic polyurethane foam

| | | | | | |
|---|---|---|---|---|---|
| | | Foam | Clear | Hazy | Total |
| Sample | Replicates | size (mm) | zone | zone | ZOI |

Staphylococcus aureus ATCC 6538

| Control foam (no | Plate -1 | 31 | No inhibition | | |
|---|---|---|---|---|---|
| treatment) | Plate -2 | 31 | | | |
| | Plate -3 | 31 | | | |
| Polylysine* | Plate -1 | 31 | 35 | 35-39 | 39 |
| | Plate -2 | 32 | 35 | 35-40 | 40 |
| | Plate -3 | 30 | 34 | 34-39 | 39 |
| Cytoguard LA 20** | Plate -1 | 29 | 36 | 36-40 | 40 |
| | Plate -2 | 30 | 35 | 35-39 | 39 |
| | Plate -3 | 30 | 32 | 32-38 | 38 |

Pseudomonas aeruginosa ATCC 9027

| Control foam (no | Plate -1 | 31 | No inhibition | | |
|---|---|---|---|---|---|
| treatment) | Plate -2 | 31 | | | |
| | Plate -3 | 31 | | | |
| Polylysine* | Plate -1 | 29 | 31 | 0 | 31 |
| | Plate -2 | 32 | 34 | 0 | 34 |
| | Plate -3 | 30 | 32 | 0 | 32 |
| Cytoguard LA 20** | Plate -1 | 31 | 32 | 0 | 32 |
| | Plate -2 | 30 | 32 | 0 | 32 |
| | Plate -3 | 28 | 29 | 0 | 29 |

Candida albicans ATCC 10231

| Control foam (no | Plate -1 | 31 | No inhibition | | |
|---|---|---|---|---|---|
| treatment) | Plate -2 | 31 | | | |
| | Plate -3 | 31 | | | |
| Polylysine* | Plate -1 | 31 | 34 | 0 | 34 |
| | Plate -2 | 30 | 34 | 0 | 34 |
| | Plate -3 | 29 | 32 | 0 | 32 |
| Cytoguard LA 20** | Plate -1 | 33 | 36 | 0 | 36 |
| | Plate -2 | 30 | 34 | 0 | 34 |
| | Plate -3 | 30 | 32 | 0 | 32 |

*Epsiliseen (Siveele B.V.) in DI water (20% solution) sprayed on foam and allowed to dry for 48 hrs; 12% polylysine on foam);
**Cytoguard LA 20 is a 10% LAE solution from A&B Ingredients; it was sprayed to foam resulting in 20% weight gain, and allowed to dry.

In addition, the antimicrobial polyurethane foam compositions were prepared by adding the LAE powder to the surfactant/polyol solution at 1.0% by weight of the final foam composition. For example, to about 14 kgs of total polyurethane foaming solution, which includes about 40% polyisocyanate, and about 140 gms (1.0%) LAE solution was added. Similarly, epsilon-polylysine at 1.0%, and Cytoguard LA20 at 3.75% were added separately. Surprisingly, the addition of polylysine, LAE, or Cytoguard LA20 to the reactant mixture did not interfere with the foaming reaction even though each of them contained free amino groups.

The antimicrobial efficacy of the foam with these agents was evaluated according to the following general procedure: The microorganisms were grown on TSA slants by incubation. Following the incubation period, the slants were washed with sterile Serological Saline Solution to harvest the microorganism. Using Culti-Loop the microorganisms were grown and adjusted to 10⁸ (CFU) colony forming units per ml and used as a stock suspension. The microbial count was adjusted to 10⁷ cfu/ml by dilution of the stock suspension. In a sterile specimen cup, 1 square inch of the foam was cut and placed. The foam sample was then inoculated with 0.2 ml of the 10⁷ cfu/ml suspension resulting in a starting CFU on the foam of 10⁶/ml. At the time intervals of Time 0, 24 hours, 72 hours, and 168 hours, 10.0 mL of sterile Serological Saline Solution was added to the specimen cup with the inoculated test product. A 1.0 ml from the specimen cup with the inoculated test product was then taken and placed into 9.0 ml of Serological Saline Solution (1:10 Dilution). Additional 1:10 serial dilutions were prepared using Serological Saline Solution to achieve 1:100 and 1:1000 dilutions. A 1.0 ml from each dilution was plated in sterile Petri dishes and melted TSA agar was added as the growth medium for bacterial organisms. The bacterial plates were incubated at 30 to 35° C. for 48 hours. The same procedure was repeated for the Serological Saline Solution control. After the incubation period, all plates were counted to determine the number of microorganisms remaining at the various time points.

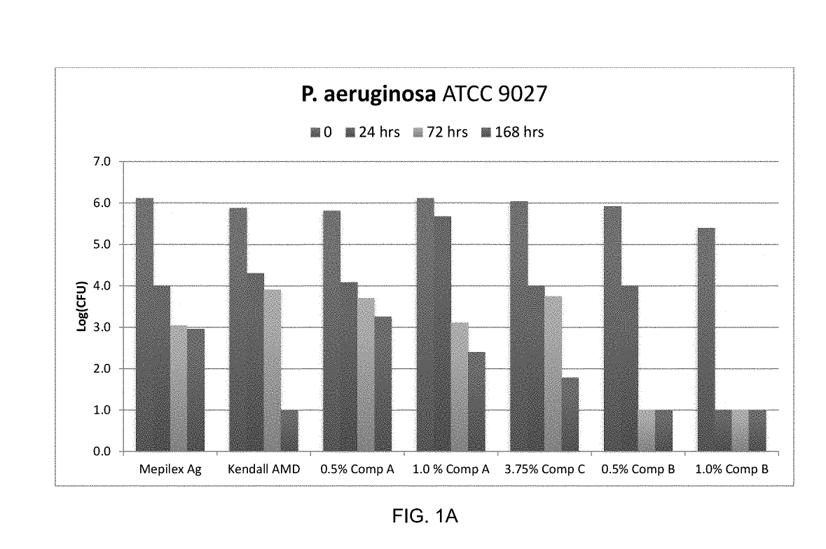

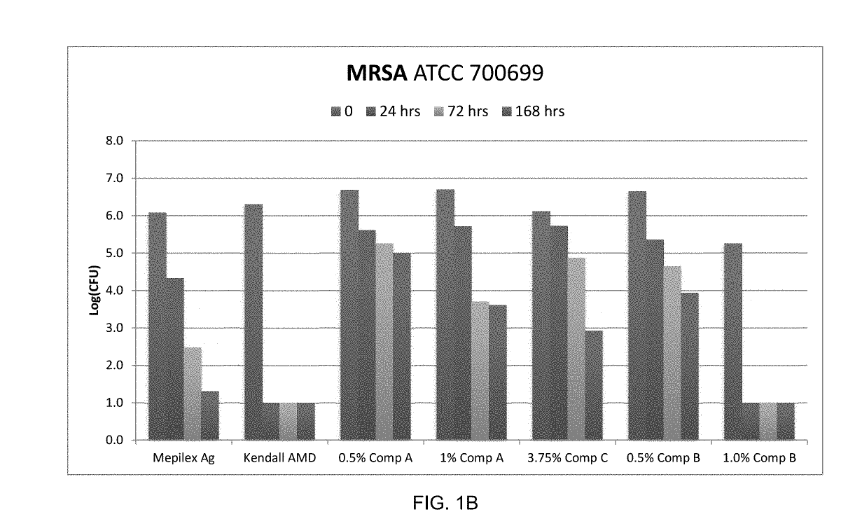

The two bacteria tested were P. aeruginosa (ATCC 9027) and MRSA (ATCC 700699). The antimicrobial foam of the present disclosure tested are as follows: Comp A: Epsiliseen (polylysine) 1%; Comp B: LAE.HCl 1%; Comp C: Cytoguard LA 20 (A&B Ingredients; 10% LAE.HCl in water-based solution) 3.75%; Commercial products: Mepilex Ag (Molnlycke AB; uses silver); Kendall AMD (Covidien; w/PHMB). It should be noted that the lower the colony forming units (CFU), the more effective the antimicrobial agent is. Results are shown in FIGS. 1A and 1B. Surprisingly, the antimicrobial foams with in situ addition of LAE.HCl were more effective than commercially available foams with PHMB or silver. This is the first time to the inventor's knowledge, the antimicrobial additives, LAE or epsilon-polylysine has been incorporated into a foaming structure in situ, and the antimicrobial effect of such novel foams are found to be superior compared to commercially available silver or PHMB-based foams.

### Example 4

### Antimicrobial Wound Gels

The wound gel can be prepared using a carrier base such as water, polyethylene glycol, propylene glycol, glycerol or other suitable liquids. At least one non-ionic thickener added to obtain the desired viscosity and consistency of gel. The gel can include preservatives for storage stability. The gel can be used to protect dry wounds such as diabetic foot ulcer. Further when the gel includes antimicrobial agents of the present disclosure, the gel can be used to reduce bio-burden in such wound environment.

For example, a 2% solution of hydroxyethylcellulose (Natrosol) was prepared to yield a gel. To 10 grams of this gel, 0.6 grams of Epsiliseen (polylysine from Siveele B. V.) was added and stirred to yield a liquid gel. To another 10 grams of the above gel, 0.6 grams of LAE (A&B Ingredients) was added and stirred to yield a liquid gel.

To evaluate the effect of different thickeners, the following formulations were made, shown in Table 8. As it can be seen, the only thickener that provided a stable gel with LAE was HEC.

TABLE 8
Effect of different thickeners with LAE•HCl

| Ingredients | A | B | C | D |
|---|---|---|---|---|
| DI water | 89.5 | 89.5 | 89.5 | 89.5 |
| PEG 200 | 5 | 5 | 5 | 5 |
| Aminat G | 3.5 | 3.5 | 3.5 | 3.5 |
| HEC | 2 | | | |
| Xanthangum | | 2 | | |
| Guar gum | | | 2 | |
| Polyacrylic acid | | | | 2 |
| Gel consistency | Clear gel | Precipitated | Hazy gel | Precipitated |

Another example shown below was prepared and tested for log-kill rate. The general procedure for the wound gel examples below are as follows: To a clean sanitized Stainless steel vessel, purified water is added, and then the Klucel or Natrosol is added slowly while mixing with a propeller at low-medium speed and the temperature of the vessel set to about 70° C. Then monolaurin, if present, is added after the Natrosol or Klucel is fully hydrated and there are no gel particles visible. Then potassium hydroxide is added and the temperature lowered to 40-45 C. After a few minutes of mixing, a pre-mix of PEG-8 and Aminat-G is added to the vessel followed by Hydrolite 5, and the temperature lowered to 25-30-deg C. After a few more minutes of mixing, the contents are transferred to a glass jar and sealed.

TABLE 9
Antimicrobial Wound gel composition

| Ingredient | Percentage |
|---|---|
| Water | 50.000 |
| PEG-8 | 35.000 |
| Klucel JF | 5.000 |
| Monolaurin | 5.000 |
| Aminat G | 2.500 |
| Hydrolite 5 | 2.500 |

The gel was testing according USP 38-2015 Antimicrobial Effectiveness Testing <51>. The results of the test are shown below. The glyceryl monolaurate is not soluble in water. Surprisingly, the present composition has incorporated the glyceryl monolaurate in a water-based gel by the use of glycols, polyethyleneglycol, and pentylene glycol.

TABLE 10
Results of antimicrobial efficacy of wound gel of Table 7

| | Preservative Testing | | | | |
|---|---|---|---|---|---|
| | Colony Forming Units/gram | | | | |
| | Inoculum/ | | | 48 | |
| Organism | g | 0 hr | 24 hrs | hrs | 72 hrs |
| Staphylococcus aureus | 1.0 × 10⁶ | <10 | <10 | <10 | <10 |
| (bacteria) (ATCC# 6538) | | | | | |
| Pseudomonas aeruginosa | 1.0 × 10⁶ | <10 | <10 | <10 | <10 |
| (bacteria) (ATCC# 9027) | | | | | |
| Candida albicans (yeast) | 1.0 × 10⁵ | <10 | <10 | <10 | <10 |
| (ATCC# 10231) | | | | | |
| Aspergillus niger (mold) | 1.0 × 10⁵ | TNTC | 80 cfu | <10 | <10 |
| (ATCC# 16404) | | | | | |

### Example 5

### Antimicrobial Wound Cleansers

The wound cleanser formulation including the component shown in Table 11 was prepared tested for antimicrobial efficacy.

The general procedure for preparing the wound cleanser is as follows: In a clean sanitized stainless steel vessel, purified water is added and propeller mixing started at med-speed. Then disodium EDTA is added mixed for about 10-15 minutes or until fully dissolved. Then the following are added one by one: Sorbitol, Crodateric CDA-40 and Polyosorbate 20 while mixing. The contents are mixed for 15 to 30 minutes. Then Aminat-G is added making sure it is fully dissolved. Then KOH solution (45%) is added to adjust the pH to between 6-7. The contents are then stored in a glass jar and sealed.

TABLE 11
Non-limiting example of wound cleanser formulation

| Ingredient | Percentage |
|---|---|
| Water | 95.568 |
| Disodium EDTA | 0.100 |
| Crodateric CDA 40 | 0.800 |
| ColaLipid C | 1.000 |
| Aminat G | 2.500 |
| KOH 45% | 0.032 |

The antimicrobial efficacy results are shown in Table 12.

TABLE 12
Antimicrobial efficacy of wound cleanser in Table 11

| | Preservative Testing | | | | |
|---|---|---|---|---|---|
| | Colony Forming Units/gram | | | | |
| | Inoculum/ | | | | 72 |
| Organism | g | 0 hr | 24 hrs | 48 hrs | hrs |
| Staphylococcus aureus | 1.0 × 10⁶ | <10 | <10 | <10 | <10 |
| (bacteria) (ATCC# 6538) | | | | | |
| Pseudomonas aeruginosa | 1.0 × 10⁶ | <10 | <10 | <10 | <10 |
| (bacteria) (ATCC# 9027) | | | | | |
| Candida albicans (yeast) | 1.0 × 10⁵ | <10 | <10 | <10 | <10 |
| (ATCC# 10231) | | | | | |
| Aspergillus niger (mold) | 1.0 × 10⁵ | TNTC | 150 cfu | 110 cfu | <10 |
| (ATCC# 16404) | | | | | |

### Example 6

### Antimicrobial Wound Dressings

A wound dressing can include at least one substrate, at least one adhesive to adhere to the wound and/or skin, wherein the adhesive may be according any one or more of the above claims. The substrate can be selected from polymer film, non-woven, woven fabric, mesh, foam, and combinations thereof. The polymer films typically used in wound care include polyurethane, polyetherblockamides, co-polyesters, polyolefins and the like. The films may be perforated or non-perforated. The non-wovens may include hydrophilic materials such as cellulose, gelatin, collagen, polyvinyl alcohol, polyurethane, etc. or non-hydrophilic non-wovens such as polyester, polyethylene, polyurethane and the like. The foams maybe open celled, reticulated, close celled, or a combination of film and foam. Typical foams include polyurethanes, polyvinyl alcohol, silicones, gelatin, cellulose, and polyethylene-vinyl acetate.

The wound dressing can be prepared by applying the adhesive to the substrate using methods known in the art of manufacturing tapes such as transfer lamination, direct coating, spray coating, and the like. The coated surface may be protected using release film layers also known as release liners. The release liners are removed prior to attached of the adhesive surface to the wound. In other aspects, the adhesive may be coated on both side of the substrate. Further, the adhesive may be coated partially or in a pattern on the substrate.

For example, a wound dressing can be prepared as follows:

Silicone gel adhesive, MG7-9900 from Dow Corning Corp. is blended with methylcellulose (Sigma Aldrich) at 95 wt % and 5wt % respectively. MG7-9900 Part A47.5 grams weighed into a plastic cup, followed by adding 47.5 grams of Part B. After mixing the two components thoroughly with a stirrer, 5 grams of methyl cellulose is added to the mixture 7-9900, and stirred thoroughly. The mixture is then coated on a polyurethane film (EU28 from Delstar) at 6 mils coating thickness using a byk-gardner knife coater. The coating is then cured at 140° C. for 5 minutes in a lab oven, then allowed to cool at room temperature before laminating a polycarbonate film to protect the adhesive surface. The MVTR of the adhesive tape (after removing the casting paper of the polyurethane film) is greater than 200 g/m²/24 hrs.

Further, it is expected that additional compositions combining two or more antimicrobial agents described herein may result in better properties of the compositions including enhanced antimicrobial effect.

### Example 7

### Additional Wound Gel Formulations and Efficacy Against E. coli and A. brasiliensis

Wound gel formulations having the composition described in Table 13 below were prepared. Results of antimicrobial efficacy testing are shown in Tables 14 and 15.

TABLE 13
Antimicrobial Wound gel formulations

| | Formulations | | |
|---|---|---|---|
| | Version 3 | Version 4 | Version 5 |
| Ingredient | Percentage | Percentage | Percentage |
| Water | 90.45 | 88.95 | 87.90 |
| Natrosol 250 HRR | 1.50 | 1.500 | 1.50 |
| KOH sol Caustic Potash | 0.05 | 0.550 | 0.59 |
| PEG 8 | 5.00 | 5.000 | 5.00 |
| Aminat G | 3.00 | 4.00 | 5.00 |
| Viscosity | 6640 cps | 8000 cps | 7920 cps |
| (Brookfield RV-DVE) | | | |
| pH | 4.7 | 4.69 | 4.68 (Initial: |
| | (initial: 6.6) | (Initial: 5.93) | 5.78) |

For E. coli, a greater than 4-log kill within 30 mins of exposure was observed. For A. brasiliensis, more 2-log reduction in 30 minutes was observed. The sustained effectiveness of the wound gels in inhibiting these specific microbes at short and long time scales is surprising, especially against the spore A. brasiliensis. It is expected that these kill rates are comparable or even better than silver or PHMB-based gels.

TABLE 14
Escherichia coli, ATCC #8739

| | Exposure | Average Control | Average Test Article | Percent | LOG₁₀ |
|---|---|---|---|---|---|
| Identification | Intervals | Titer (CFH/ml) | Titer (CFU/ml) | Reduction (%) | Reduction |
| Control | 1 hour | 1.7 × 10⁶ | 1.6 × 10⁶ | 6.0 | 0.03 |
| JE160831-3 | 30 minutes | | <2.0 × 10¹ | >99.9988 | >4.93 |
| | 24 hours | | <2.0 × 10¹ | >99.9988 | >4.93 |
| | 72 hours | | <2.0 × 10¹ | >99.9988 | >4.93 |
| | 168 hours | | <2.0 × 10¹ | >99.9988 | >4.93 |
| JE160831-4 | 30 minutes | | <2.0 × 10¹ | >99.9988 | >4.93 |
| | 24 hours | | <2.0 × 10¹ | >99.9988 | >4.93 |
| | 72 hours | | <2.0 × 10¹ | >99.9988 | >4.93 |
| | 168 hours | | <2.0 × 10¹ | >99.9988 | >4.93 |
| JE160831-5 | 30 minutes | | <2.0 × 10¹ | >99.9988 | >4.93 |
| | 24 hours | | <2.0 × 10¹ | >99.9988 | >4.93 |
| | 72 hours | | <2.0 × 10¹ | >99.9988 | >4.93 |
| | 168 hours | | <2.0 × 10¹ | >99.9988 | >4.93 |

TABLE 15
Aspergillus brasiliensis, ATCC #16404

| | Exposure | Average Control | Average Test Article | Percent | LOG₁₀ |
|---|---|---|---|---|---|
| Identification | Intervals | Titer (CFH/ml) | Titer (CFU/ml) | Reduction (%) | Reduction |
| Control | 1 hour | ~1.7 × 10⁶ | 8.3 × 10⁵ | ~50 | ~0.30 |
| JE160831-3 | 30 minutes | | ~4.3 × 10³ | ~99.74 | ~2.59 |
| | 24 hours | | ~3.3 × 10¹ | ~99.9980 | ~4.70 |
| | 72 hours | | <2.0 × 10¹ | ~99.9988 | ~4.92 |
| | 168 hours | | <2.0 × 10¹ | ~99.9988 | ~4.92 |
| JE160831-4 | 30 minutes | | ~5.4 × 10³ | ~99.68 | ~2.49 |
| | 24 hours | | ~2.7 × 10¹ | ~99.9984 | ~4.80 |
| | 72 hours | | <2.0 × 10¹ | ~99.9988 | ~4.92 |
| | 168 hours | | <2.0 × 10¹ | ~99.9988 | ~4.92 |
| JE160831-5 | 30 minutes | | ~2.3 × 10⁴ | ~98.6 | ~1.85 |
| | 24 hours | | ~2.7 × 10¹ | ~99.9984 | ~4.80 |
| | 72 hours | | <2.0 × 10¹ | ~99.9988 | ~4.92 |
| | 168 hours | | <2.0 × 10¹ | ~99.9988 | ~4.92 |

### Example 8

### Hydrophilic Silicone Adhesive Composition

The hydrophilic silicone adhesive having the following composition was prepared: silicone gel adhesive (Blend of Part A+Part B): 85% by weight; Glycerol: 10% by weight; Hydroxyethyl cellulose: 5% by weight.

The above composition was mixed, coated, and cured on polyurethane film at 140-150° C., and the resulting cured adhesive surface protected with a release liner. Silicone gel adhesive is formed as a resulted of reaction between Part A (vinyl-containing polydimethylsiloxane polymer with platinum catalyst) and Part B (blend of hydride-containing polydimethylsiloxane polymer (cross-linker) and vinyl-polydimethylsiloxane polymer) components.

The hydrophilic silicone adhesive composition had a peel strength of 35 g/in against polycarbonate substrate, and MVTR of 750-900 gms/m²/24 hrs. A neat silicone gel without the hydrophilic additives, had an MVTR of 150-200 gms/m²/24 hrs.

### Example 9

### Wound Cleanser

A wound cleanser was prepared having the components shown below (wherein the percentages are by weight). The mix procedure outlined in Example 5 are applicable here.

| Ingredient | % (by weight) |
|---|---|
| Purified water | 95.850 |
| Disodium EDTA | 0.050 |
| Sorbitol | 1.500 |
| Crodateric CDA 40 | 0.500 (lipid-based surfactant) |
| Polysorbate 20 | 0.100 |
| TEA 98% | QS |
| Aminat G | 0.1-3.5 (or LAE level: 0.02 g-0.7 g) |

The Aminat G or LAE level can be adjusted depending on desired effect, for example, for antibacterial effect, lower levels are required; and for antimicrobial effect, higher levels are required.

### Example 10

### Additional Wound Cleanser Formulations and Efficacy Against E. coli and A. brasiliensis

The following wound cleansers examples were prepared according to the following general procedure outlined in Example 5 above.

The time-kill antimicrobial efficacy studies were conducted per ASTM E 2315-03. 2008 Standard Guide for Assessment of Antimicrobial Activity using a Time-Kill Procedure, and Clinical and Laboratory Standards Institute, Vol 19 No. 18, 1999. M26-A. Methods for Determining Bactericidal Activity of Antimicrobial Agents: Approved Guideline.

E. coli is a gram negative bacteria and Aspergillus brasiliensis is a spore.

TABLE 16
Antimicrobial Wound Cleanser I formulations

| Wound Cleanser I | JE160822-1 | JE160822-2 | JE160822-3 |
|---|---|---|---|
| Ingredient | Percentage | Percentage | Percentage |
| Water | | | |
| Disodium EDTA | 0.05 | 0.05 | 0.050 |
| Sorbitol 70% | 1.500 | 1.500 | 1.500 |
| Crodateric CDA-40 | 0.500 | 0.500 | 0.500 |
| Polysorbate-20 | 0.100 | 0.100 | 0.100 |
| Aminat G | 2.000 | 3.500 | 5.000 |
| Potassium Hydroxyde sol | 0.045 | 0.060 | 0.064 |
| Initial pH before KOH Sol | 4.65 | 4.35 | 4.280 |
| Final pH | 6.68 | 6.89 | 6.590 |
| LAE % | 0.4 | 0.7 | 1.0 |

TABLE 17
Antimicrobial Wound Cleanser II formulations

| | JE160824- | JE160824- | JE160824- | JE160824- |
|---|---|---|---|---|
| Wound Cleanser II | 1 | 2 | 3 | 4 |
| Ingredient | Percentage | Percentage | Percentage | Percentage |
| Water | 95.649 | | 96.399 | 95.949 |
| Aloe Vera Powder 200X | 0.001 | 0.001 | 0.001 | 0.001 |
| Tetrasodium EDTA | 0.300 | 0.300 | 0.300 | N/A |
| Premix | | | | |
| Propylene Glycol | 1.000 | 0.500 | 1.000 | 1.000 |
| Coco-Betaine | 1.500 | 1.500 | 1.500 | 1.500 |
| Endilan E-51 | 0.500 | 0.500 | 0.500 | 0.500 |
| Citric Acid QS to pH | 0.050 | 0.050 | 0.050 | |
| Aminat G | 1.000 | 0.500 | 0.250 | 1.000 |
| KOH sol | | | | 0.050 |
| Initial pH before | 9.97 | 9.95 | 9.980 | 5.800 |
| Citric Acid or KOH | | | | |
| Final pH | 6.83 | 6.9 | 6.910 | 6.870 |
| LAE % | 0.2 | 0.10 | 0.05 | 0.2 |

Results: Wound Cleanser I and II—Efficacy Against E. coli and A. brasiliensis

Results for both, Cleansers I and II, against E. coli and A. brasiliensis are shown in Tables 18, 19, 20 and 21. It can be seen that even at low levels of 0.05 and 0.1%, the compositions are highly effective in reducing the microbes, greater than 4-log kill with E. coli within 1 min. of exposure, and greater than 70% reduction with A. brasiliensis within a minute of exposure. This is surprising because of the minimum inhibitory concentration (MIC) for pure LAE is 16 micrograms per mL for E. coli, and 32 micrograms per mL for A. brasiliensis. At about 15 times or higher than the MIC, the present cleanser formulations show fast kill rates and also sustained kill rates over time. For an antimicrobial wound cleanser to be effective, it needs to provide biocidal or antimicrobial effect at short exposure times and continue the effect over time. The cleanser compositions of the present disclosure are expected to adhere to the wound layer thereby delivering the LAE on the wound site for prolonged antimicrobial effect.

TABLE 18
Wound Cleanser I: Efficacy against Escherichia coli, ATCC #8739

| | | Average | Average Test | | |
|---|---|---|---|---|---|
| | Exposure | Control Titer | ArticleTiter | Percent | LOG₁₀ |
| Identification | Intervals | (CFH/ml) | (CFU/ml) | Reduction (%) | Reduction |
| Control | 1 hour | 1.7 × 10⁶ | 1.6 × 10⁶ | 6.0 | 0.03 |
| JE160822-1 | 10 minutes | | <2.0 × 10¹ | >99.9988 | >4.93 |
| | 30 minutes | | <2.0 × 10¹ | >99.9988 | >4.93 |
| | 120 minutes | | <2.0 × 10¹ | >99.9988 | >4.93 |
| | 24 hours | | <2.0 × 10¹ | >99.9988 | >4.93 |
| JE160822-2 | 10 minutes | | <2.0 × 10¹ | >99.9988 | >4.93 |
| | 30 minutes | | <2.0 × 10¹ | >99.9988 | >4.93 |
| | 120 minutes | | <2.0 × 10¹ | >99.9988 | >4.93 |
| | 24 hours | | <2.0 × 10¹ | >99.9988 | >4.93 |
| JE160822-3 | 10 minutes | | <2.0 × 10¹ | >99.9988 | >4.93 |
| | 30 minutes | | <2.0 × 10¹ | >99.9988 | >4.93 |
| | 120 minutes | | <2.0 × 10¹ | >99.9988 | >4.93 |
| | 24 hours | | <2.0 × 10¹ | >99.9988 | >4.93 |

TABLE 19
Wound Cleanser I: Efficacy against Aspergillus brasiliensis, ATCC #16404

| | | Average | Average Test | | |
|---|---|---|---|---|---|
| | Exposure | Control Titer | Article Titer | Percent | LOG₁₀ |
| Identification | Intervals | (CFH/ml) | (CFU/ml) | Reduction (%) | Reduction |
| Control | 1 hour | ~1.7 × 10⁶ | 8.3 × 10⁵ | ~50 | ~0.30 |
| JE160822-1 | 10 minutes | | ~3.2 × 10⁵ | ~81 | ~0.72 |
| | 30 minutes | | ~2.2 × 10⁵ | ~87 | ~0.88 |
| | 120 minutes | | <2.7 × 10⁴ | ~98.4 | ~1.79 |
| | 24 hours | | <2.0 × 10² | ~99.988 | ~3.92 |
| JE160822-2 | 10 minutes | | ~2.1 × 10⁵ | ~87 | ~0.89 |
| | 30 minutes | | ~4.5 × 10⁴ | ~97.3 | ~1.57 |
| | 120 minutes | | ~3.4 × 10³ | ~99.8 | ~2.69 |
| | 24 hours | | <2.0 × 10¹ | ~99.9988 | ~4.92 |
| JE160822-3 | 10 minutes | | 6.3 × 10⁴ | ~96.2 | ~1.42 |
| | 30 minutes | | ~2.6 × 10⁴ | ~98.4 | ~1.81 |
| | 120 minutes | | ~5.1 × 10² | ~99.970 | ~3.52 |
| | 24 hours | | <2.0 × 10¹ | ~99.9988 | ~4.92 |

Wound Cleanser II—Efficacy Against E. coli and A. brasiliensis

With E. coli, greater than 4-log kill within 10 mins of exposure, and with A. brasiliensis, there is a 1-log reduction in 120 minutes, which is surprising. The results are shown in Tables 20 and 21 below.

TABLE 20
Wound Cleanser II: Efficacy against Escherichia coli, ATCC #8739

| | | | Average | | |
|---|---|---|---|---|---|
| | | Average | Test | | |
| | | Control | Article | Percent | |
| | Exposure | Titer | Titer | Reduction | LOG₁₀ |
| Identification | Intervals | (CFH/ml) | (CFU/ml) | (%) | Reduction |
| Control | 5 mins | 1.7 × 10⁶ | 1.5 × 10⁶ | 15 | 0.07 |
| | 1 hour | | 1.6 × 10⁶ | 6 | 0.03 |
| JE160824-1 | 1 minute | | <2.0 × 10¹ | >99.9988 | >4.93 |
| | 2 minutes | | <2.0 × 10¹ | >99.9988 | >4.93 |
| | 5 minutes | | <2.0 × 10¹ | >99.9988 | >4.93 |
| JE160824-2 | 1 minute | | <2.0 × 10¹ | >99.9988 | >4.93 |
| | 2 minutes | | <2.0 × 10¹ | >99.9988 | >4.93 |
| | 5 minutes | | <2.0 × 10¹ | >99.9988 | >4.93 |
| JE160824-3 | 1 minute | | <2.0 × 10¹ | >99.9988 | >4.93 |
| | 2 minutes | | <2.0 × 10¹ | >99.9988 | >4.93 |
| | 5 minutes | | <2.0 × 10¹ | >99.9988 | >4.93 |
| JE160824-4 | 1 minute | | <2.0 × 10¹ | >99.9988 | >4.93 |
| | 2 minutes | | <2.0 × 10¹ | >99.9988 | >4.93 |
| | 5 minutes | | <2.0 × 10¹ | >99.9988 | >4.93 |

TABLE 21
Wound Cleanser II: Efficacy against Aspergillus brasiliensis, ATCC #16404

| | | Average | Average Test | | |
|---|---|---|---|---|---|
| | Exposure | Control Titer | Article Titer | Percent | LOG₁₀ |
| Identification | Intervals | (CFH/ml) | (CFU/ml) | Reduction (%) | Reduction |
| Control | 5 mins | ~1.7 × 10⁶ | 1.8 × 10⁶ | ~−8 | ~−0.03 |
| | 1 hour | | ~8.3 × 10⁵ | ~50 | ~0.3 |
| JE160824-1 | 1 minute | | ~3.1 × 10⁵ | ~82 | ~0.74 |
| | 2 minutes | | <4.0 × 10⁵ | ~76 | ~0.62 |
| | 5 minutes | | <4.0 × 10⁵ | ~76 | ~0.62 |
| JE160822-2 | 1 minute | | ~3.1 × 10⁵ | ~81 | ~0.73 |
| | 2 minutes | | ~4.4 × 10⁵ | ~74 | ~0.58 |
| | 5 minutes | | ~5.3 × 10⁵ | ~68 | ~0.50 |
| JE160822-3 | 1 minute | | <3.5 × 10⁵ | ~79 | ~0.68 |
| | 2 minutes | | ~5.4 × 10⁵ | ~68 | ~0.49 |
| | 5 minutes | | ~5.0 × 10⁵ | ~70 | ~0.52 |
| JE160824-4 | 1 minute | | ~3.3 × 10⁵ | ~80 | ~0.71 |
| | 2 minutes | | ~4.5 × 10⁵ | ~73 | ~0.57 |
| | 5 minutes | | ~4.9 × 10⁵ | ~71 | ~0.53 |

### Example 11

### Skin and Tissue Substitute/Scaffold Dressing

A 25% gelatin solution is prepared, and then desired amount of lauroyl arginate ethyl ester salt and/or polylysine Epsiliseen-H is added as a solution or powder to the gelatin solution. The mixture is then extruded from a syringe into fibers into a container which rotates at 4500 rpm or so as described in US 2010/0285291 A1 and US 2015/0010612 A1 referenced herewith in their entireties. The fabric or fleece made using such process when tested for antimicrobial efficacy according to the present disclosure is expected to have a zone of inhibition as the size of the fleece or fabric, and a log-reduction of at least one-order of magnitude against S. aureus and P. aeruginosa.

As a non-limiting example, an aqueous or non-aqueous solution or suspension or emulsion containing LAE or polylysine may be applied to the skin or tissue substitute or scaffold by spraying or brushing or other suitable techniques of application. The antimicrobial treated dressing when tested for antimicrobial efficacy according to the present disclosure is expected to have a zone of inhibition as the size of the fleece or fabric, and a log-reduction of at least one-order of magnitude against S. aureus and P. aeruginosa.

While the above specification includes many specifics and details of the disclosure, these should not be construed as limitations on the scope of the disclosure, but rather as examples of aspects of the disclosure. Additional combinations and various compositions are possible, as can be inferred by one skilled in the art. The scope of the disclosure should be determined not by the illustrated aspects, but by the appended claims and their legal equivalents.

Unless otherwise indicated, all numbers and values expressing quantities, concentrations, amounts, percentages, and so forth, as used herein are to be understood as being modified by the term “about.”

While this invention has been particularly shown and described with references to preferred embodiments thereof, it will be understood by those skilled in the art that various changes in form and details may be made therein without departing from the scope of the invention encompassed by the appended claims.

1-51. (canceled)

52. An antimicrobial wound gel comprising:
a. N^{α}-lauroyl-arginine ethyl ester or a salt thereof in an amount between about 0.05 to about 5% by weight of the composition; and
b. a non-ionic thickener selected from the group consisting of hydroxyethylcellulose, hydroxypropyl cellulose, methyl cellulose, and polyethylene oxide in an amount between about 0.5 to about 5% by weight of the composition;
wherein the wound gel is an aqueous gel with a viscosity greater than 1,000 centipoise.

53. The wound gel of claim 52, further comprising polyethylene glycol.

54. The wound gel of claim 52, wherein the non-ionic thickener is selected from the group consisting of hydroxyethylcellulose, hydroxypropyl cellulose, and methyl cellulose.

55. The wound gel of claim 54, wherein the non-ionic thickener is hydroxyethyl cellulose or hydroxypropyl cellulose.

56. The wound gel of claim 55, wherein the non-ionic thickener is hydroxyethyl cellulose.

57. The wound gel of claim 52, comprising the components of the table below:

| Component | Amount (% by weight of composition) |
|---|---|
| PEG 8 | 5 |
| Hydroxyethylcellulose | 2 |
| LAE | 0.7 |

58. The wound gel of claim 57, further comprising a buffer.

59. The wound gel of claim 52, wherein the composition does not comprise polyhexamethylene biguanide (PHMB), hypochlorous acid, silver and salts thereof, chlorhexidine gluconate, iodine, hypochlorous acid and/or octenidine dihydrochloride.

60. The wound gel of claim 52, wherein the gel is skin-safe.

61. A method of treating a wound in a subject in need thereof, wherein the wound is at risk for infection and/or biofilm formation or regrowth, comprising treating the wound with the wound gel of claim 52.

62. A method for treating a burn, scar, bacterial infection, viral infection, and/or fungal infection in a subject in need thereof comprising treating the affected area with the wound gel of claim 52.

63-180. (canceled)

181. The wound gel of claim 52, wherein the N^{α}-lauroyl-arginine ethyl ester or a salt thereof is in an amount between about 0.05 to about 3% by weight of the composition.

182. The wound gel of claim 52, wherein the gel can reduce the number of colony forming units (CFU) of a microbe by at least one order of magnitude in 24 hours of exposure.

183. The wound gel of claim 182, wherein the number of colony forming units (CFUs) of Staphylococcus aureus are reduced by at least one order of magnitude in 24 hours of exposure.

184. The wound gel of claim 182, wherein the number of colony forming units (CFUs) of Pseudomonas aeruginosa are reduced by at least one order of magnitude in 24 hours of exposure.
